SEQUENCE LISTING (72 sequences)

SEQ ID NO: 1 - RNA, 21 nt, Artificial Sequence
  siRNA to human fidgetin-like 2, sense strand
uuacacaguauuaaagcgauu

SEQ ID NO: 2 - RNA, 21 nt, Artificial Sequence
  siRNA to human fidgetin-like 2, antisense strand
ucgcuuuaauacuguguaauu

SEQ ID NO: 3 - RNA, 21 nt, Artificial Sequence
  siRNA to human fidgetin-like 2, sense strand
caucugaaaccuagggucuuu

SEQ ID NO: 4 - RNA, 21 nt, Artificial Sequence
  siRNA to human fidgetin-like 2, antisense strand
agacccuagguuucagauguu

SEQ ID NO: 5 - RNA, 21 nt, Artificial Sequence
  siRNA to human fidgetin-like 2, sense strand
gugacuuaugcuaggaggauu

SEQ ID NO: 6 - RNA, 21 nt, Artificial Sequence
  siRNA to human fidgetin-like 2, antisense strand
uccuccuagcauaagucacuu

SEQ ID NO: 7 - RNA, 21 nt, Artificial Sequence
  siRNA to human fidgetin-like 2, sense strand
ggucagaagcagaauguauuu

SEQ ID NO: 8 - RNA, 21 nt, Artificial Sequence
  siRNA to human fidgetin-like 2, antisense strand
auacauucugcuucugaccuu

SEQ ID NO: 9 - RNA, 21 nt, Artificial Sequence
  siRNA to human fidgetin-like 2, sense strand
  misc_feature (20)..(20): n is deoxythymidine
  misc_feature (21)..(21): n is deoxythymidine
cgccggcccacaaguuggann

SEQ ID NO: 10 - RNA, 21 nt, Artificial Sequence
  siRNA to human fidgetin-like 2, antisense strand
  misc_feature (20)..(20): n is deoxythymidine
  misc_feature (21)..(21): n is deoxythymidine
uccaacuugugggccggcgnn

SEQ ID NO: 11-16 - skipped (6 entries)

SEQ ID NO: 17 - DNA, 21 bp, Artificial Sequence
  siRNA to human fidgetin-like 2, sense strand
  misc_feature (1)..(1): n is 2′-fluoruridine
  misc_feature (2)..(2): n is 2′-fluorouridine
  misc_feature (3)..(3): n is 2′-O-methyladenosine
  misc_feature (4)..(4): n is 2′fluorocytidine
  misc_feature (5)..(5): n is 2′O-methyladenosine
  misc_feature (6)..(6): n is 2′fluorocytidine
  misc_feature (6)..(6): n is 2′fluorodeoxycytidine
nnnnnnaguauuaaagcgatt

SEQ ID NO: 18 - DNA, 21 bp, Artificial Sequence
  siRNA to human fidgetin-like 2, antisense strand 3′ end has a phosphodiester cap
ttaaugugucauaauuucgcu

SEQ ID NO: 19 - DNA, 4711 bp, Human
agtgagctatggggacactactgcactgtagcctgggcaacagagcaagaccttgtctcaaaaatgtatatatattttgggctttttttcctaaaacgggaactacaacagcatatttgcgagctgatgagagtgacccagcagagagggaaatggatcagctctgttgaagatgcactggacaccagaacacgcccagcccctcaaccagtggccagagcagcacctggacgtctcctccaccaccccgtcgccggcccacaagttggagttgccccctgggggtcgccaacgctgccactacgcttgggcacacgacgacatctcagccctcactgcctccaacctcctaaagcgctatgcagagaagtactctggggtcttggattctccctacgagcgtccggccctgggcgggtacagcgacgcctccttcctcaacggcgccaaaggggatcccgagccctggccagggccggagccaccctaccccttggcctcactccacgaaggcctcccaggaaccaaatcgggcggtggcggcggttccggggccctggggggctccccagttttagccgggaacctccctgaacccctctacgccggcaatgcgtgcgggggcccatcggcggcgcccgagtacgcggccggctacggcggggggtacctggcgccgggttactgcgcgcagacgggcgccgcgctgcccccgccgcccccggccgcgctcctgcagcccccaccgcctccggggtacgggccctcagcgccgctgtacaactatcccgcagggggctacgcagcgcagcccggctatggcgcgctcccgccgcccccaggcccacccccggccccctacctgaccccgggcctgcccgcgcccacgcccctgcccgcgccggcaccgcccaccgcctatggcttccccacggccgcgccgggtgccgaatccgggctgtcgctgaagcgcaaggccgccgacgaggggcccgagggccgctaccgcaagtacgcgtacgagcccgccaaggcccccgtggctgacggagcctcctaccccgccgcggacaacggcgaatgtcggggcaacgggttccgggccaagccgccaggagccgcggaggaggcgtcgggcaagtacggtggcggcgtccccctcaaggtcctgggctcccccgtctacggcccgcaactggagccctttgaaaagttcccggagcgggccccggctcctcgtggggggttcgccgtgccgtcgggggagactcccaaaggcgtggaccctggggccctggagctggtgacgagcaagatggtggactgcgggcccccggtgcagtgggcggatgtggcgggccagggcgcgctcaaggcggcgctggaggaggagctggtgtggcccctgctcaggccgcccgcctacccgggcagcctgcgcccgccgcggaccgtcctgctctttgggccgcggggcgcgggcaaagcgctgctgggccgctgcctcgccacgcagctgggcgccacgctgttgcgcctgcgcggcgcgaccctggctgcgcccggcgccgccgagggcgcgcgcctcctccaggccgccttcgcggccgcgcgctgccgcccaccctccgtactcctcatcagcgagctagaggcgctgctccccgcccgggacgacggcgcggcggcagggggcgcgctgcaggtgccgctcctggcctgcctggacgggggctgcggcgcgggggctgacggcgtgctggttgtgggcaccacctcgcggcccgcggctctggacgaggcgacccgccggcgcttctctctccgcttctacgtggcgctgcccgacagcccggcccgcgggcagatcctgcagcgggcgctggcccagcagggctgcgcgctcagtgagcgggaactggcggcgctggtgcagggcacgcagggcttctctgggggcgagctggggcagctgtgccagcaggcggcggccggggcgggcctcccggggctgcagcgccccctctcctacaaggacctggaggcggcgctggccaaggtgggccctagggcctctgccaaggaactggactcgttcgtggagtgggacaaaatgtacggctccggacactgacggcgcgcgggggaggccgcgggagccgcagtccctccgtccccgccgcctccgcgtgggagggatgtcactgactaaacccggctggcaggggctggagtggtgaatgtgggatcggggacaggaggggtctgccggtggatattttttttttcgtgggaaggaaaatgcttctgccaggcagatgccatatgcgccgtgtactcaggtttttcctatttattgtggactggaagctcgccatctccgcccggcagaccgggcagatccggcatgggctggcacccggggccttaagaactcctgctctcttgccacaacgcttttgtctcctcgctatctgaatggcaccctccttctccctcactctctccatcccattctctgcattctcttggttttctctcccttttgctttgtcgctgacacccctgcccaccccatgctggccctgtttctctcctgcccctccctccccagctctccatccctcaccctctgtgcttctgtctccatccctggctctccagcgtccctggccttttggtccctgagctttaatgcctttccctgccttctgttcttatttggactgcagtggccctttgcaggagctctggaggcccaggggctgaggaggagggttacccctctacccatctgaaacctagggtctagggggatcaaggaaaaaaagtccccaaagaaggggaattttttgtttgtttttgaggggagatcccagaaatgtagcttgtttcatattttagtcttcttatttttgtaaaatgtgtagaatttgctgtttttctttttcttttgacaactcaggaagaaactgacctcagaaagaatgttagactttggctgctctcctgtgtgcccctcacacctgccccctcccccccactccatccaggggaccaaattctcccagacactcaaaaaatgagacttacggggaaggggagaggaagacccagaggcctcagtgaaaccccagctattcctggtcagaagcagaatgtattcctaagggcttcctccccagggccgaggcctaggcatgaatgtggggagtgggctgtggggtttgagagaagggaggccttattcctctcctgctgctccccaccccctgccccacccaacccctccgctgagtgttttctgtgaagggctatccagagttaggatgcccttgcccaattccttcctgagacccagaaggtagggtgggagggcccaaatgggaaggtgacctaagcagaaagtctccagaaaggtcatgtcccctggccctgccttggcagaggtccccagtgacttatgctaggaggattccatctgggtagacagtctggccacaaaatcagctactggacctcagccatctctgctggaggctctgaggaggagtgagcatccctcacttgtgggggctctgtgaggaaatgtgccttccccattcccccggagtcctaggtctggagctccagggctgggagagggtgagggagatgggcaggggtgttttctctgaccttgggggcttagtctcagtcctgcctgaactttccactaggcttggaacccttccaagaaccatatttctctccttcccaccaattttcccttgatgaggctttagcagtttgctcccaccacccccagcccatttcacaactctgatcttagtccaaagcaggggacacgcccccccaccaccactttttctctctcccatctcagcctcctgtgcagttccttgcctgcccgtgcatttcctagagtctactgcctcccccctggctgggagggtgtctgggggggatctttcaggggccctggcacccagggcctgtgctggcctaggagtgctgaccagaaggctgctctgttcccccccacccccgttgctttctggccccctctttggagccagccacccacagggctttggtgcctcagaagcagtgggctgccgggtcacagccgcaggctgcaaaagaccctcggagggagcatggagtgaggggttctctctcaggtgtgtatgtattggggggtgggggtgggtggagggtgtcagggaagttggggtgggatcccagccttcccttcaagaggcagggagctctgggaggtggagtccccaccgctttctctactaggctcctcctgttccccaggcttggggagctttgcacaaggagactgcccccagcctagtggcacctacctcatgggctctggggcaggtaggggaagggccagtccagctctggtaatgctggggggaggcataccaaagaatccaggggcagggagtggggagggtgacttccgagctggcctctccccttcctctacccagactggggctgggatcctctcctcccgctgtaaccatttctacctcattttgctgcgtgttgtacatggacgtatttatctcctgtctgacgatgctctgcagttgtggtctgtctacctcagaagagactgtattttaaaagaaagtattacacagtattaaagcgatgacatgtggtttgcaaaaaaaaaaaaaaaaaa

SEQ ID NO: 20 - protein, 653 aa, Human
MHWTPEHAQPLNQWPEQHLDVSSTTPSPAHKLELPPGGRQRCHYAWAHDDISALTASNLLKRYAEKYSGVLDSPYERPALGGYSDASFLNGAKGDPEPWPGPEPPYPLASLHEGLPGTKSGGGGGSGALGGSPVLAGNLPEPLYAGNACGGPSAAPEYAAGYGGGYLAPGYCAQTGAALPPPPPAALLQPPPPPGYGPSAPLYNYPAGGYAAQPGYGALPPPPGPPPAPYLTPGLPAPTPLPAPAPPTAYGFPTAAPGAESGLSLKRKAADEGPEGRYRKYAYEPAKAPVADGASYPAADNGECRGNGFRAKPPGAAEEASGKYGGGVPLKVLGSPVYGPQLEPFEKFPERAPAPRGGFAVPSGETPKGVDPGALELVTSKMVDCGPPVQWADVAGQGALKAALEEELVWPLLRPPAYPGSLRPPRTVLLFGPRGAGKALLGRCLATQLGATLLRLRGATLAAPGAAEGARLLQAAFAAARCRPPSVLLISELEALLPARDDGAAAGGALQVPLLACLDGGCGAGADGVLVVGTTSRPAALDEATRRRFSLRFYVALPDSPARGQILQRALAQQGCALSERELAALVQGTQGFSGGELGQLCQQAAAGAGLPGLQRPLSYKDLEAALAKVGPRASAKELDSFVEWDKMYGSGH

SEQ ID NO: 21-33 - skipped (13 entries)

SEQ ID NO: 34 - DNA, 21 bp, Artificial Sequence
  siRNA to human fidgetin-like 2, sense strand
uuacacaguauuaaagcgatt

SEQ ID NO: 35 - DNA, 21 bp, Artificial Sequence
  siRNA to human fidgetin-like 2, antisense strand 5′ phosphodiester cap
  misc_feature (1)..(1): n is 2′-O-methyluridine
  misc_feature (2)..(2): n is 2′O′methylcytidine
nngcuuuaauacuguguaatt

SEQ ID NO: 36 - DNA, 21 bp, Artificial Sequence
  siRNA to human fidgetin-like 2, sense strand 5′ phosphodiester cap Phosphorothioate bond between nucleotides 1 and 2 and between nucleotides 2 and 3
  misc_feature (1)..(1): n is 2′-O′methyuridine
  misc_feature (1)..(2): phosphorotioate linkage
  misc_feature (1)..(1): 5 phosphodiester cap
  misc_feature (2)..(3): phosphorotioate linkage
  misc_feature (2)..(2): n is 2′-O-methylcytidine
nngcuuuaauacuguguaatt

SEQ ID NO: 37 - DNA, 21 bp, Artificial Sequence
  siRNA to human fidgetin-like 2, antisense strand 5′ phosphodiester cap
  misc_feature (1)..(1): n is 2′-fluorodeoxyuridine
  misc_feature (1)..(1): 5′ phosphodiester cap
  misc_feature (2)..(2): n is 2′-fluorodeoxycytidine
nngcuuuaauacuguguaatt

SEQ ID NO: 38 - DNA, 21 bp, Artificial Sequence
  siRNA to human fidgetin-like 2, sense strand 5′ phosphodiester cap Phosphorothioate bond between nucleotides 1 and 2 and between nucleotides 2 and 3
  misc_feature (1)..(1): 2′-fluorodeoxyuridine
  misc_feature (1)..(1): 5′ phosphodiester cap
  misc_feature (1)..(2): phosphorothioate bond
  misc_feature (2)..(3): phosphorothioate bond
  misc_feature (2)..(2): 2′-fluorodeoxycytidine
nngcuuuaauacuguguaatt

SEQ ID NO: 39 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3
  misc_feature (1)..(1): 1 n is 2′-O-methyluridine
  misc_feature (1)..(1): 5′ phosphodiester cap
  misc_feature (1)..(2): phosphorothioate bond
  misc_feature (2)..(3): phosphorothioate bond
  misc_feature (2)..(2): n is 2′-O-methylcytidine
  misc_feature (12)..(12): n is 2′-O-methylcytidine
  misc_feature (13)..(13): n is 2′-fluorodeoxyuridine
  misc_feature (14)..(14): n is 2′-O-methylguanosine
  misc_feature (15)..(15): n is 2′-fluorodeoxyuridine
  misc_feature (16)..(16): n is 2′-O-methylguanosine
  misc_feature (17)..(17): n is 2′-fluorodeoxyuridine
  misc_feature (18)..(18): n is 2′-O′-methyladenosine
  misc_feature (19)..(19): n is 2′-O′-methyladenosine
nngcuuuaauannnnnnnntt

SEQ ID NO: 40 - DNA, 21 bp, Artificial Sequence
  siRNA to human fidgetin-like 2, sense strand 5′ phosphodiester cap Phosphorothioate bond between nucleotides 1 and 2
  misc_feature (1)..(1): 5′ phosphodiester cap
  misc_feature (1)..(2): phosphorotioate bond
ucgcuuuaauacuguguaatt

SEQ ID NO: 41 - DNA, 21 bp, Artificial Sequence
  siRNA to human fidgetin-like 2, antisense strand 5′ phosphodiester cap
  misc_feature (1)..(1): n is 2′-O-methyluridine
  misc_feature (1)..(1): 5′ phosphodiester cap
  misc_feature (2)..(2): n is 2′-fluorodeoxycytidine
  misc_feature (3)..(3): n is 2′-O-methylguanosine
  misc_feature (4)..(4): n is 2′-fluorodeoxycytidine
  misc_feature (5)..(5): n is 2′-O-methyluridine
  misc_feature (6)..(6): n is 2′-fluorodeoxyuridine
  misc_feature (7)..(7): n is 2′-O-methyluridine
  misc_feature (10)..(10): n is 2′-fluorodeoxyuridine
  misc_feature (11)..(11): n is 2′-O′-methyladenosine
  misc_feature (12)..(12): n is 2′-fluorodeoxycytidine
  misc_feature (13)..(13): n is 2′-O-methyluridine
  misc_feature (15)..(15): n is 2′-O-methyluridine
  misc_feature (16)..(16): n is 2′-O-methylguanosine
  misc_feature (17)..(17): n is 2′-fluorodeoxyuridine
  misc_feature (18)..(18): n is 2′-O′-methyladenosine
  misc_feature (19)..(19): n is 2′-O′-methyladenosine
nnnnnnnaannnngnnnnntt

SEQ ID NO: 42 - RNA, 21 nt, Artificial Sequence
  siRNA to human fidgetin-like 2, sense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is 2′-O-methylcytidine
  misc_feature (18)..(18): n is 2′-O-methylguanosine
  misc_feature (19)..(19): n is 2′-O′-methyladenosine
  misc_feature (20)..(20): n is 2′-O-methyluridine
  misc_feature (21)..(21): n is 2′-O-methyluridine
nnnnnnnnnnnnnnnnnnnnn

SEQ ID NO: 43 - RNA, 20 nt, Artificial Sequence
  siRNA to human fidgetin-like 2, antisense strand 5′ phosphodiester cap
  misc_feature (1)..(1): n is 2′-O-methyluridine
  misc_feature (1)..(1): 5′ phosphodiester cap
  misc_feature (2)..(2): n is 2′-O-methylcytidine
  misc_feature (3)..(3): n is 2′-O-methylguanosine
  misc_feature (4)..(4): n is 2′-O-methylcytidine
  misc_feature (5)..(5): n is 2′-O-methyluridine
  misc_feature (6)..(6): n is 2′-O-methyluridine
  misc_feature (7)..(7): n is 2′-O-methyluridine
  misc_feature (8)..(8): n is 2′-O′-methyladenosine
  misc_feature (9)..(9): n is 2′-O′-methyladenosine
  misc_feature (10)..(10): n is 2′-O-methyluridine
  misc_feature (11)..(11): n is 2′-O′-methyladenosine
  misc_feature (12)..(12): n is 2′-O-methylcytidine
  misc_feature (13)..(13): n is 2′-O-methyluridine
  misc_feature (14)..(14): n is 2′-O-methylguanosine
  misc_feature (15)..(15): n is 2′-O-methyluridine
  misc_feature (16)..(16): n is 2′-O-methylguanosine
  misc_feature (17)..(17): n is 2′-O-methyluridine
  misc_feature (18)..(18): n is 2′-O′-methyladenosine
  misc_feature (19)..(19): n is 2′-O′-methyladenosine
  misc_feature (20)..(20): n is 2′-O-methyluridine
nnnnnnnnnnnnnnnnnnnn

SEQ ID NO: 44 - DNA, 21 bp, Artificial Sequence
  siRNA to human fidgetin-like 2, sense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is deoxycytidine
  misc_feature (18)..(18): n is deoxyguanosine
  misc_feature (19)..(19): n is deoxyadenosine
nnnnnnnnnnnnnnnnnnntt

SEQ ID NO: 45 - DNA, 21 bp, Artificial Sequence
  siRNA to human fidgetin-like 2, antisense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is deoxycytidine
  misc_feature (18)..(18): n is 2′-O-methylguanosine
  misc_feature (19)..(19): n is 2′-O′-methyladenosine
nnnnnnnnnnnnnnnnnnntt

SEQ ID NO: 46 - RNA, 19 nt, Artificial Sequence
  siRNA to human fidgetin-like 2, sense strand
uuacacaguauuaaagcga

SEQ ID NO: 47 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): 5′ phosphodiester cap
  misc_feature (1)..(2): phosphorothioate bond
ucgcuuuaauacuguguaatt

SEQ ID NO: 48 - DNA, 21 bp, Artificial Sequence
  siRNA to human fidgetin-like 2, sense strand 5′ phosphodiester cap
  misc_feature (1)..(1): 5′ phosphodiester cap
ucgcuuuaauacuguguaatt

SEQ ID NO: 49 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3
  misc_feature (1)..(1): 5′ phosphodiester cap
  misc_feature (1)..(2): phosphorothioate bond
  misc_feature (2)..(3): phosphorothioate bond
ucgcuuuaauacuguguaatt

SEQ ID NO: 50 - RNA, 19 nt, Artificial Sequence
  siRNA to human fidgetin-like 2, sense strand
  misc_feature (1)..(1): n is 2′-O-methyluridine
  misc_feature (2)..(2): n is 2′-O-methyluridine
nnacacaguauuaaagcga

SEQ ID NO: 51 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): 5′ phosphodiester cap
  misc_feature (1)..(2): phosphorothioate bond
  misc_feature (18)..(18): n is 2′-O′-methyladenosine
  misc_feature (19)..(19): n is 2′-O′-methyladenosine
ucgcuuuaauacugugunntt

SEQ ID NO: 52 - DNA, 21 bp, Artificial Sequence
  siRNA to human fidgetin-like 2, sense strand 5′ phosphodiester cap
  misc_feature (1)..(1): 5′ phosphodiester cap
ucgcuuuaauacuguguaatt

SEQ ID NO: 53 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3 and between nucleotides 19 and 20
  misc_feature (1)..(1): 5′ phosphodiester cap
  misc_feature (1)..(2): phosphorothioate bond
  misc_feature (2)..(3): phosphorothioate bond
  misc_feature (19)..(20): phosphorothioate bond
ucgcuuuaauacuguguaatt

SEQ ID NO: 54 - DNA, 21 bp, Artificial Sequence
  siRNA to human fidgetin-like 2, sense strand
  misc_feature (1)..(1): n is locked uridine
  misc_feature (2)..(2): n is locked uridine
  misc_feature (3)..(3): n is locked adenosine
  misc_feature (4)..(4): n is locked cytidine
  misc_feature (5)..(5): n is locked adenosine
nnnnncaguauuaaagcgatt

SEQ ID NO: 55 - DNA, 21 bp, Artificial Sequence
  siRNA to human fidgetin-like 2, antisense strand 5′ phosphodiester cap
  misc_feature (1)..(1): 5′ phosphodiester cap
  misc_feature (15)..(15): n is locked uridine
  misc_feature (16)..(16): n is locked guanosine
  misc_feature (17)..(17): n is locked uridine
  misc_feature (18)..(18): n is locked adenosine
  misc_feature (19)..(19): n is locked adenosine
ucgcuuuaauacugnnnnntt

SEQ ID NO: 56 - RNA, 19 nt, Artificial Sequence
  siRNA to human fidgetin-like 2, sense strand
  misc_feature (1)..(1): n is 2′-fluorodeoxyuridine
  misc_feature (2)..(2): n is 2′-fluorodeoxyuridine
  misc_feature (3)..(3): n is locked adenosine
  misc_feature (4)..(4): n is 2′-fluorodeoxycytidine
  misc_feature (5)..(5): n is locked adenosine
nnnnncaguauuaaagcga

SEQ ID NO: 57 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): n is 2′-O-methyluridine
  misc_feature (1)..(1): 5′ phosphodiester cap
  misc_feature (1)..(2): phosphorothioate bond
  misc_feature (2)..(2): n is 2′-O-methylcytidine
  misc_feature (3)..(3): n is 2′-O-methylguanosine
nnncuuuaauacuguguaatt

SEQ ID NO: 58 - DNA, 21 bp, Artificial Sequence
  siRNA to human fidgetin-like 2, antisense strand
  misc_feature (1)..(1): n is 2′-fluorodeoxydeoxyuridine
  misc_feature (2)..(2): n is 2′-fluorodeoxydeoxycytidine
nngcuuuaauacuguguaatt

SEQ ID NO: 59 - DNA, 21 bp, Artificial Sequence
  iRNA to human fidgetin-like 2, antisense strand Phosphorothioate bond between nucleotides 1 and 2 and between nucleotides 2 and 3
  misc_feature (1)..(1): n is 2′-fluorodeoxydeoxyuridine
  misc_feature (2)..(2): n is 2′-fluorodeoxydeoxycytidine
nngcuuuaauacuguguaatt

SEQ ID NO: 60 - DNA, 21 bp, Artificial Sequence
  siRNA to human fidgetin-like 2, antisense strand Phosphorothioate bond between nucleotides 1 and 2 and between nucleotides 2 and 3
  misc_feature (1)..(1): 1 n is 2′-O-methyluridine
  misc_feature (2)..(2): n is 2′-O-methylcytidine
  misc_feature (12)..(12): n is 2′-O-methylcytidine
  misc_feature (13)..(13): n is 2′-fluorodeoxydeoxyuridine
  misc_feature (14)..(14): n is 2′-O-methylguanosine
  misc_feature (15)..(15): n is 2′-fluorodeoxydeoxyuridine
  misc_feature (16)..(16): n is 2′-O-methylguanosine
  misc_feature (17)..(17): n is 2′-fluorodeoxydeoxyuridine
  misc_feature (18)..(18): n is 2′-O-methyladenosine
  misc_feature (19)..(19): n is 2′-O-methyladenosine
nngcuuuaauannnnnnnntt

SEQ ID NO: 61 - DNA, 21 bp, Artificial Sequence
  siRNA to human fidgetin-like 2, antisense strand Phosphorothioate bond between nucleotides 1 and 2
ucgcuuuaauacuguguaatt

SEQ ID NO: 62 - DNA, 21 bp, Artificial Sequence
  siRNA to human fidgetin-like 2, antisense strand
  misc_feature (1)..(1): n is 2′-O-methyluridine
  misc_feature (2)..(2): n is 2′-fluorodeoxydeoxycytidine
  misc_feature (3)..(3): n is 2′-O-methylguanosine
  misc_feature (4)..(4): n is 2′-fluorodeoxydeoxycytidine
  misc_feature (5)..(5): n is 2′-O-methyluridine
  misc_feature (6)..(6): n is 2′-fluorodeoxydeoxyuridine
  misc_feature (7)..(7): n is 2′-O-methyluridine
  misc_feature (10)..(10): n is 2′-fluorodeoxydeoxyuridine
  misc_feature (11)..(11): n is 2′-O-methyladenosine
  misc_feature (12)..(12): n is 2′-fluorodeoxydeoxycytidine
  misc_feature (13)..(13): n is 2′-O-methyluridine
  misc_feature (15)..(15): n is 2′-O-methyluridine
  misc_feature (16)..(16): n is 2′-O-methylguanosine
  misc_feature (17)..(17): n is 2′-fluorodeoxydeoxyuridine
  misc_feature (18)..(18): n is 2′-O-methyladenosine
  misc_feature (19)..(19): n is 2′-O-methyladenosine
nnnnnnnaannnngnnnnntt

SEQ ID NO: 63 - DNA, 20 bp, Artificial Sequence
  siRNA to human fidgetin-like 2, antisense strand
  misc_feature (1)..(1): n is 2′-O-methyluridine
  misc_feature (2)..(2): n is 2′-O-methylcytidine
  misc_feature (3)..(3): n is 2′-O-methylguanosine
  misc_feature (4)..(4): n is 2′-O-methylcytidine
  misc_feature (5)..(5): n is 2′-O-methyluridine
  misc_feature (6)..(6): n is 2′-O-methyluridine
  misc_feature (7)..(7): n is 2′-O-methyluridine
  misc_feature (8)..(8): n is 2′-O-methyladenosine
  misc_feature (9)..(9): n is 2′-O-methyladenosine
  misc_feature (10)..(10): n is 2′-O-methyluridine
  misc_feature (11)..(11): n is 2′-O-methyladenosine
  misc_feature (12)..(12): n is 2′-O-methylcytidine
  misc_feature (13)..(13): n is 2′-O-methyluridine
  misc_feature (14)..(15): n is a, c, g, or t
  misc_feature (16)..(16): n is 2′-O-methylguanosine
  misc_feature (17)..(17): n is 2′-O-methyluridine
  misc_feature (18)..(18): n is 2′-O-methyladenosine
  misc_feature (19)..(19): n is 2′-O-methyladenosine
  misc_feature (20)..(20): n is 2′-O-methyluridine
nnnnnnnnnnnnnnnnnnnn

SEQ ID NO: 64 - DNA, 21 bp, Artificial Sequence
  siRNA to human fidgetin-like 2, antisense strand Phosphorothioate bond between nucleotides 1 and 2
ucgcuuuaauacuguguaatt

SEQ ID NO: 65 - DNA, 21 bp, Artificial Sequence
  siRNA to human fidgetin-like 2, antisense strand
ucgcuuuaauacuguguaatt

SEQ ID NO: 66 - DNA, 21 bp, Artificial Sequence
  siRNA to human fidgetin-like 2, antisense strand Phosphorothioate bond between nucleotides 1 and 2 and between nucleotides 2 and 3
ucgcuuuaauacuguguaatt

SEQ ID NO: 67 - DNA, 21 bp, Artificial Sequence
  siRNA to human fidgetin-like 2, antisense strand Phosphorothioate bond between nucleotides 1 and 2
  misc_feature (18)..(18): n is 2′-O-methyladenosine
  misc_feature (19)..(19): n is 2′-O-methyladenosine
ucgcuuuaauacugugunntt

SEQ ID NO: 68 - DNA, 21 bp, Artificial Sequence
  siRNA to human fidgetin-like 2, antisense strand
ucgcuuuaauacuguguaatt

SEQ ID NO: 69 - DNA, 21 bp, Artificial Sequence
  siRNA to human fidgetin-like 2, antisense strand Phosphorothioate bond between nucleotides 1 and 2 and between nucleotides 2 and 3 and between nucleotides 19 and 20
ucgcuuuaauacuguguaatt

SEQ ID NO: 70 - DNA, 21 bp, Artificial Sequence
  siRNA to human fidgetin-like 2, antisense strand
  misc_feature (15)..(15): n is locked uridine
  misc_feature (16)..(16): n is locked guanosine
  misc_feature (17)..(17): n is locked uridine
  misc_feature (18)..(18): n is locked adenosine
  misc_feature (19)..(19): n is locked adenosine
ucgcuuuaauacugnnnnntt

SEQ ID NO: 71 - DNA, 21 bp, Artificial Sequence
  siRNA to human fidgetin-like 2, antisense strand Phosphorothioate bond between nucleotides 1 and 2
  misc_feature (1)..(1): n is 2′-O-methyluridine
  misc_feature (2)..(2): n is 2′-O-methylcytidine
  misc_feature (3)..(3): n is 2′-O-methylguanosine
nnncuuuaauacuguguaatt

SEQ ID NO: 72 - DNA, 21 bp, Artificial Sequence
  siRNA to human fidgetin-like 2, antisense strand
ucgcuuuaauacuguguaatt

(19) United States
(12) Patent Application Publication
(10) Pub. No.: US 2024/0294918 A1
(43) Pub. Date: Sep. 5, 2024

(54) FIDGETIN-LIKE 2 AS A TARGET TO ENHANCE WOUND HEALING

(71) Applicant: MICROCURES, INC., Santa Cruz, CA (US)

(72) Inventors: Douglas G. BROOKS, Carlsbad, CA (US); Brian O'ROURKE, Rockville Centre, NY (US); Adam KRAMER, Queens, NY (US); David James SHARP, Scarsdale, NY (US)

(73) Assignee: MICROCURES, INC., Santa Cruz, CA (US)

(21) Appl. No.: 17/802,916

(22) PCT Filed: Feb. 27, 2021
(86) PCT No.: PCT/US21/20130

§ 371 Date: Aug. 26, 2022

Related U.S. Application Data
(60) Provisional application No. 62/983,193, filed on Feb. 28, 2020.

(51) Int. Cl. C12N 15/113; A61K 9/107; A61K 9/70; A61K 47/06; A61K 47/10; A61K 47/20; A61K 47/32; A61K 47/34; A61K 47/36; A61K 47/42; A61P 17/02

(52) U.S. Cl. CPC C12N 15/1137; A61K 9/1075; A61K 9/7007; A61K 47/06; A61K 47/10; A61K 47/20; A61K 47/32; A61K 47/34; A61K 47/36; A61K 47/42; A61P 17/02; C12N 2310/14; C12N 2310/315; C12N 2310/321

(57) ABSTRACT

Nucleic acid molecules and compositions thereof targeted to fidgetin-like 2, and methods of their use in treating a wound or scar in a human subject are described.

### CROSS-REFERENCE

This application claims priority to U.S. Provisional Patent Application 62/983,193, filed Feb. 28, 2020, which is incorporated by reference herein in its entirety.

### INCORPORATION OF SEQUENCE LISTING

The “.txt” Sequence Listing filed with this application by EFS and which is entitled P-592953-US-SQL-updated-29MAR23_ST25.txt, is 44.1 kilobytes in size and which was created on Mar. 29, 2023, is hereby incorporated by reference. The sequence listing submitted herewith is identical to the sequence listing forming part of the international application.

### BACKGROUND

The development of safe and effective therapies for treating acute and chronic wounds is an issue currently of great interest to clinical scientists and industry, alike. Wound healing is an intricate, multi-stage process that relies heavily on the delivery of new cells to the wound zone. Two key elements of the wound healing response are fibroplasia and epithelialization when fibroblasts and epithelial cells, respectively, enter the wound to form a protective barrier from the external environment. This is stimulated by cell proliferation and migration from the wound edge. The identification of agents that increase the rate at which cells invade and close a wound would represent a major advance in wound healing therapeutics. Ideally, this would be a topically or locally applied agent that stimulates the proliferation and migration of fibroblasts and wound edge epithelial cells.

The disclosures of all publications, patents, patent application publications and books referred to in this application are hereby incorporated by reference in their entirety into the subject application to more fully describe the art to which the present disclosure pertains.

### SUMMARY

In one aspect, a nucleic acid molecule is provided consisting of a sequence selected from the group consisting of:

(SEQ ID NO: 17)
5′-fUfUmA fCmAfC AGU AUU AAA GCG ATT;
(SEQ ID NO: 18)
(Phos) 5′-U CGC UUU AAU ACU G UG UAA TT;
(SEQ ID NO: 34)
5′-UUACACAGUAUUAAAGCGATT-3′;
(SEQ ID NO: 35)
(Phos) 5′-mUmCGCUUUAAUACUGUGUAATT-3′;
(SEQ ID NO: 36)
(Phos) 5′-mU(s)mC(s)GCUUUAAUACUGUGUAATT-3′;
(SEQ ID NO: 37)
(Phos) 5′-fUfCGCUUUAAUACUGUGUAATT-3′;
(SEQ ID NO: 38)
(Phos) 5′-fU(s)fC(s)GCUUUAAUACUGUGUAATT-3′;
(SEQ ID NO: 39)
(Phos) 5′-mU(s)mC(s)GCUUUAAUAmCfUmGfUmGfUmAmAT
T-3′;
(SEQ ID NO: 40)
(Phos) 5′-U(s)CGCUUUAAUACUGUGUAATT-3′;
(SEQ ID NO: 41)
(Phos) 5′-mUfCmGfCmUfUmUAAfUmAfCmUGmUmGfUmAmATT;
(SEQ ID NO: 42)
5′-mUmUmAmCmAmCmAmGmUmAmUmUmAmAmAmGmCmGmAmUmU-3′;
(SEQ ID NO: 43)
(Phos) 5′-mUmCmGmCmUmUmUmAmAmUmAmCmUmGmUmGmUmAmAm
UmU-3′;
(SEQ ID NO: 44)
5′ mUmUmAmCmAmCmAmGmUmAmUmUmAmAmAmGdCdGdATT-3′;
(SEQ ID NO: 45)
5′ mUmUmAmCmAmCmAmGmUmAmUmUmAmAmAmGdCmGmATT-3′;
(SEQ ID NO: 46)
5′ UUACACAGUAUUAAAGCGA-3′;
(SEQ ID NO: 47)
(Phos) 5′-U(s)CGCUUUAAUACUGUGUAATT-3′;
(SEQ ID NO: 48)
(Phos) 5′-UCGCUUUAAUACUGUGUAATT-3′;
(SEQ ID NO: 49)
(Phos) 5′-U(s)C(s)GCUUUAAUACUGUGUAATT-3′;
(SEQ ID NO: 50)
5′-mUmUACACAGUAUUAAAGCGA-3′;
(SEQ ID NO: 51)
(Phos) 5′-U(s)CGCUUUAAUACUGUGUmAmATT-3′;
(SEQ ID NO: 52)
(Phos) 5′-UCGCUUUAAUACUGUGUAATT-3′;
(SEQ ID NO: 53)
(Phos) 5′-U(s)C(s)GCUUUAAUACUGUGUAA T(s)T-3′;
(SEQ ID NO: 54)
5′ lUlUlAlClACAGUAUUAAAGCGATT-3′;
SEQ ID NO: 55)
(Phos) 5′-UCGCUUUAAUACUGlUlGlUlAlA TT-3′;
(SEQ ID NO: 56)
5′ fUfUlAfClACAGUAUUAAAGCGA-3′;
or
(SEQ ID NO: 57)
(Phos) 5′-mU(s)mCmGCUUUAAUACUGUGUAATT-3′,
wherein d(nucleotide) = deoxy-(nucleotide),
m(nucleotide) = 2′-O-methyl nucleotide, T =
thymidine, f(nucleotide) = 2′-fluorodeoxy
nucleotide, (Phos) = phosphodiester cap; capital
letter nucleotide = RNA nucleotide,
l(nucleotide) = a locked nucleotide, and (s) =
phosphorothioate. For example, in SEQ ID NO: 17,
fC represents 2′-fluorodeoxy cytidine
ribonucleic acid, fU represents 2′-fluorodeoxy
uracil ribonucleic acid, and mA represents
2′-O-methyl adenosine ribonucleic acid.

In some embodiments, any of the foregoing sequences shown with a phosphodiester cap may be provided without a phosphodiester cap, such as SEQ ID NOs:58-72 described herein.

In some embodiments, the siRNA consists of any of the foregoing sequences. In some embodiments, the siRNA comprises of any of the foregoing sequences. In some embodiments, a double stranded nucleic acid is provided consisting of two nucleic acid molecules selected from among SEQ ID NOs: 17-18 and 34-72. In some embodiments, a double stranded nucleic acid is provided comprising at least one nucleic acid molecule selected from among SEQ ID NOs: 17-18 and 34-72.

In some embodiments, the siRNA has at least one modification selected from a 3′ overhang, a 5′ overhang, a 5′ phosphorylation, a 2′ sugar modification, a nucleic acid base modification, a phosphate backbone modification, and any combination of any of the foregoing. Any of the siRNA sequences may have a phosphodiester cap. In some embodiments, any of the foregoing sequences shown with a phosphodiester cap may be provided without a phosphodiester cap, such as SEQ ID NOs:58-72 described herein.

In some embodiments, a double stranded nucleic acid is provided consisting of an antisense nucleic acid molecule and a sense nucleic acid molecule, each selected from among SEQ ID NOs: 17-18 and 34-72.

In some embodiments, a double stranded nucleic acid is provided comprising an antisense nucleic acid molecule selected from among SEQ ID NOs: 17-18 and 34-72, and a sense nucleic acid molecule selected from among SEQ ID NOs: 17-18 and 34-72.

In some embodiments, a double stranded nucleic acid is provided comprising two nucleic acid molecules selected from among SEQ ID NOs: 17-18 and 34-72. In some embodiments, the double stranded nucleic acid comprises a sense strand and an antisense strand.

In some embodiments, each strand of the double stranded nucleic acid has no more than 52 nucleotides.

In some embodiments, a double-stranded nucleic acid is provided consisting of SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; or SEQ ID NO:56 and SEQ ID NO:57.

In some embodiments, any one of the foregoing nucleic acids has at least one nucleotide is modified or further modified. In some embodiments, the modified nucleotide is selected from 2′-O-methyl-adenosine, 2′-O-methyl-uridine, 2′-O-methyl-cytosine, 2′-O-methyl-guanosine, 2′-O-methyl-thymidine, 2′-fluoro-adenosine, 2′-fluoro-cytidine, 2′-fluoro-guanosine, 2′-fluoro-uracil, 2′-fluoro-thymidine, deoxycytosine, deoxyguanosine, deoxyadenosine, deoxythymidine, deoxyuridine, a locked adenosine, a locked uridine, a locked guanosine, a locked cytidine, a phosphorothioate, and a phosphodiester cap. In some embodiments, at least one additional nucleotide or modified nucleotide is added to an end of the nucleic acid.

In one aspect, a composition is provided comprising any of the foregoing nucleic acid molecules or double-stranded nucleic acids, and a pharmaceutically acceptable carrier, vehicle, excipient or diluent.

In some embodiments, the carrier comprises at least one of the following: saline, a sugar, a polypeptide, a polymer, a lipid, a cream, a gel, a micelle material, a wafer and a nanoparticle. In some embodiments, the carrier comprises at least one of the following: a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a nucleic acid delivery vehicle, a ligand functionalized-hydrophilic polymer grafted polymer, and a ligand functionalized liposome. In some embodiments, the carrier comprises a cationic polymer-nucleic acid complex. In some embodiments, the hydrophilic polymer is polyethylene glycol (PEG).

In some embodiments, the carries comprises collagen. In some embodiments the composition is collagen microparticles. In some embodiments the nucleic acid molecule is adsorbed to the collagen.

In some embodiments, the nanoparticle is a liposomal nanoparticle. In some embodiments, the liposome is further functionalized with at least one 2′ sugar modification.

In one aspect, a method of treating a wound or inhibiting, reducing or preventing a scar in a subject is provided comprising administering to the subject a therapeutically effective amount of any of the foregoing compositions. In some embodiments, the wound or scar is of the skin, eye, central nervous system, peripheral nervous system, cardiac tissue, blood vessel, tendon, ligament, muscle, oral cavity, lips, palate, internal organs, surgical wounds, abdominal cavity, pelvic cavity or thoracic cavity. In some embodiments, the wound or scar of the eye is of the cornea or lens capsule. In some embodiments, the wound or scar results from eye surgery, LASIK surgery, LASEK surgery, PRK surgery, glaucoma filtration surgery, cataract surgery, and corneal cicatrisation.

In some embodiments, inhibition of scarring reduces the number of incidences of adhesion formation and/or the size of adhesions formed. In some embodiments, the where the prevention, reduction or inhibition of scarring enhances neuronal reconnection and/or neuronal function. In some embodiments, the cardiac tissue wound is from a myocardial infraction. In some embodiments, the wound is a neuronal wound. In some embodiments, the wound results in a capsular contraction. In some embodiments, the wound is a surgical wound. In some embodiments, the wound is from a cosmetic procedure or a scar revision. In some embodiments, skin graft healing is enhanced using a composition of the disclosure.

### BRIEF DESCRIPTION OF THE DRAWINGS

FIG. 1 shows the cycle of steps involved in the solid phase synthesis of the sense and antisense strands of SEQ ID NOs: 17/18 API.

FIG. 2 depicts the SEQ ID NO:17/18 API Manufacturing Scheme.

FIG. 3A depicts the structure of the sense strand SEQ ID NO:17.

FIG. 3B depicts the structure of the antisense strand SEQ ID NO:18.

FIG. 4 shows that siRNA-mediated depletion of FL2 enhances cell migration.

FIG. 5 shows the results of an initial scratch test screen of siRNAs for migration using U2OS cells.

FIG. 6 shows a second scratch test screen of siRNAs for migration using U2OS cells.

FIG. 7 shows a Western blot for FL2 using cells from the study of FIG. 6.

FIG. 8 depicts the results of a time-lapse scratch test using SEQ ID NO:17/18.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In some embodiments, the mammal to be treated is human. The human can be any human of any age. In an embodiment, the human is an adult. In another embodiment, the human is a child. The human can be male, female, pregnant, middle-aged, adolescent, or elderly. According to any of the methods of the present disclosure and in some embodiments, the subject is human. In another embodiment, the subject is a non-human primate. In another embodiment, the subject is murine, which in some embodiments is a mouse, and, in another embodiment is a rat. In another embodiment, the subject is canine, feline, bovine, equine, laprine or porcine. In another embodiment, the subject is mammalian. As will be noted herein, treatment of a non-human animals (e.g., non-human primate, non-human mammal) using the teachings of the disclosure may require use of a siRNAs directed to the orthologue of fidgetin-like 2 in the particular species.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

The present disclosure is directed to nucleic acid sequences that inhibit human fidgetin-like 2 activity, pharmaceutical compositions thereof, and methods of their use for preventing or treating various injuries, wounds and diseases.

### Nucleic Acid Sequences

In some embodiments, the disclosure is directed to a nucleic acid molecule consisting of one of the following sequences:

Sense strand
(SEQ ID NO: 17)
5′-fUfUmAfCmAfCAGUAUUAAAGCGATT;
Antisense strand:
(SEQ ID NO: 18)
(Phos) 5′-U CGC UUU AAU ACU G UG UAA TT;
Sense strand:
(SEQ ID NO: 34)
5′-UUACACAGUAUUAAAGCGATT-3′;
Antisense strand:
(SEQ ID NO: 35)
(Phos) 5′-mUmCGCUUUAAUACUGUGUAATT-3′;
Antisense strand:
(SEQ ID NO: 36)
(Phos) 5′-mU(s)mC(s)GCUUUAAUACUGUGUAATT-3′;
Antisense strand:
(SEQ ID NO: 37)
(Phos) 5′-fUfCGCUUUAAUACUGUGUAATT-3′;
Antisense strand:
(SEQ ID NO: 38)
(Phos) 5′-fU(s)fC(s)GCUUUAAUACUGUGUAATT-3′;
Antisense strand:
(SEQ ID NO: 39)
(Phos) 5′-mU(s)mC(s)GCUUUAAUAmCfUmGfUmGfUmAmAT
T-3′;
Antisense strand:
(SEQ ID NO: 40)
(Phos) 5′-U(s)CGCUUUAAUACUGUGUAATT-3′;
Antisense strand:
(SEQ ID NO: 41)
(Phos) 5′-mUfCmGfCmUfUmUAAfUmAfCmUGmUmGfUmAmAT
T-3′;
Sense strand:
(SEQ ID NO: 42)
5′-mUmUmAmCmAmCmAmGmUmAmUmUmAmAmAmGmCmGmAmUmU-3′;
Antisense strand:
(SEQ ID NO: 43)
(Phos) 5′-mUmCmGmCmUmUmUmAmAmUmAmCmUmGmUmGmUmAmAm
UmU-3′;
Sense strand:
(SEQ ID NO: 44)
5′-mUmUmAmCmAmCmAmGmUmAmUmUmAmAmAmGdCdGdATT-3′;
Sense strand:
(SEQ ID NO: 54)
5′-mUmUmAmCmAmCmAmGmUmAmUmUmAmAmAmGdCmGmATT-3′;
Sense strand:
(SEQ ID NO: 46)
5′-UUACACAGUAUUAAAGCGA-3′;
Antisense strand:
(SEQ ID NO: 47)
(Phos) 5′-U(s)CGCUUUAAUACUGUGUAATT-3′;
Antisense strand:
(SEQ ID NO: 48)
(Phos) 5′-UCGCUUUAAUACUGUGUAATT-3′;
Antisense strand:
(SEQ ID NO: 49)
(Phos) 5′-U(s)C(s)GCUUUAAUACUGUGUAATT-3′;
Sense strand:
(SEQ ID NO: 50)
5′-mUmUACACAGUAUUAAAGCGA-3′;
Antisense strand:
(SEQ ID NO: 51)
(Phos) 5′-U(s)CGCUUUAAUACUGUGUmAmATT-3′;
Antisense strand:
(SEQ ID NO: 52)
(Phos) 5′-UCGCUUUAAUACUGUGUAATT-3′;
Antisense strand:
(SEQ ID NO: 53)
(Phos) 5′-U(s)C(s)GCUUUAAUACUGUGUAA T(s)T-3′;
Sense strand:
(SEQ ID NO: 54)
5′-lUlUlAlClACAGUAUUAAAGCGATT-3′;
Antisense strand:
(SEQ ID NO: 55)
(Phos) 5′-UCGCUUUAAUACUGlUlGlUlAlA TT-3′;
Sense strand:
(SEQ ID NO: 56)
5′-fUfUlAfClACAGUAUUAAAGCGA-3′
Antisense strand:
(SEQ ID NO: 57)
(Phos) 5′-mU(s)mCmGCUUUAAUACUGUGUAATT-3′;
Antisense strand:
(SEQ ID NO: 58)
5′-fUfCGCUUUAAUACUGUGUAATT-3′;
Antisense strand:
(SEQ ID NO: 59)
5′-fU(s)fC(s)GCUUUAAUACUGUGUAATT-3′;
Antisense strand:
(SEQ ID NO: 60)
5′-mU(s)mC(s)GCUUUAAUAmCfUmGfUmGfUmAmATT-3′;
Antisense strand:
(SEQ ID NO: 61)
5′-U(s)CGCUUUAAUACUGUGUAATT-3′;
Antisense strand:
(SEQ ID NO: 62)
5′-mUfCmGfCmUfUmUAAfUmAfCmUGmUmGfUmAmATT;
Antisense strand:
(SEQ ID NO: 63)
5′- mUmCmGmCmUmUmUmAmAmUmAmCmUmGmUmGmUmAmAmUm
U-3′;
Antisense strand:
(SEQ ID NO: 64)
5′-U(s)CGCUUUAAUACUGUGUAATT-3′;
Antisense strand:
(SEQ ID NO: 65)
5′-UCGCUUUAAUACUGUGUAATT-3′;
Antisense strand:
(SEQ ID NO: 66)
5′-U(s)C(s)GCUUUAAUACUGUGUAATT-3′;
Antisense strand:
(SEQ ID NO: 67)
5′-U(s)CGCUUUAAUACUGUGUmAmATT-3′;
Antisense strand:
(SEQ ID NO: 68)
5′-UCGCUUUAAUACUGUGUAATT-3′;
Antisense strand:
(SEQ ID NO: 69)
5′-U(s)C(s)GCUUUAAUACUGUGUAA T(s)T-3′;
Antisense strand:
(SEQ ID NO: 70)
5′-UCGCUUUAAUACUGlUlGlUlAlA TT-3′;
Antisense strand:
(SEQ ID NO: 71)
5′-mU(s)mCmGCUUUAAUACUGUGUAATT-3′;
or
Antisense strand:
(SEQ ID NO: 72)
5′-U CGC UUU AAU ACU G UG UAA TT;
and a complement;
wherein d(nucleotide) = deoxy-(nucleotide),
m(nucleotide) = 2′-O-methyl nucleotide, T =
thymidine, f(nucleotide) = 2′-fluorodeoxy
nucleotide, (Phos) = phosphodiester cap;
capital letter nucleotide = RNA nucleotide,
l(nucleotide) = a locked nucleotide, and
(s) = phosphorothioate.

Thus, for example dT represents deoxythymidine, dC represents deoxycytidine, fC represents 2′-fluorodeoxy cytidine ribonucleic acid, fU represents 2′-fluorodeoxy uracil ribonucleic acid, mA represents 2′-O-methyl adenosine ribonucleic acid, mU represents 2′-O-methyl uracil ribonucleic acid, mC represents 2′-O-methyl cytosine ribonucleic acid, and mG represents 2′-O-methyl guanosine ribonucleic acid.

In some embodiments, complement refers to the complementary nucleic acid strand comprising a double-stranded nucleic acid. In some embodiments, if a sense strand is selected, its complement is an antisense strand. In some embodiments if an antisense strand is selected, its complement is a sense strand.

In some embodiments, the complement may be selected from any of SEQ ID NO:17-18 and 34-72. In some embodiments, if the siRNA molecule is a sense strand from among SEQ ID NOs: 17-18 and 34-72, the complement may be selected from an antisense strand from among SEQ ID NOs: 17-18 and 34-72. In some embodiments, if the siRNA molecule is an antisense strand from among SEQ ID NOs: 17-18 and 34-72, the complement may be selected from a sense strand from among SEQ ID NOs: 17-18 and 34-72.

In some embodiments, the complement may be selected from SEQ ID NOs:1-10. In some embodiments, if the siRNA molecule is a sense strand from among SEQ ID NOs: 17-18 and 34-72, the complement may be selected from an antisense strand from among SEQ ID NOs:1-10. In some embodiments, if the siRNA molecule is an antisense strand from among SEQ ID NOs: 17-18 and 34-72, the complement may be selected from a sense strand from among SEQ ID NOs:1-10. SEQ ID NOs:1-10 are: Sense strand: UUACACAGUAUUAAAGCGAUU (SEQ ID NO:1); Antisense strand: 5′ UCGCUUUAAUACUGUGUAAUU (SEQ ID NO:2); Sense strand: CAUCUGAAACCUAGGGUCUUU (SEQ ID NO:3); Antisense strand: 5′ AGACCCUAGGUUUCAGAUGUU (SEQ ID NO:4); Sense strand: GUGACUUAUGCUAGGAGGAUU (SEQ ID NO:5); Antisense strand: 5′UCCUCCUAGCAUAAGUCACUU (SEQ ID NO:6); Sense strand: GGUCAGAAGCAGAAUGUAUUU (SEQ ID NO:7); Antisense strand: 5′ AUACAUUCUGCUUCUGACCUU (SEQ ID NO:8); Sense: 5′ CGCCGGCCCACAAGUUGGAdTdT (SEQ ID NO:9); and Antisense: 5′ UCCAACUUGUGGGCCGGCGdTdT (SEQ ID NO:10).

In some embodiment, any of the nucleic acid sequences disclosed herein may be modified or further modified with one or more nucleotide modifications as described herein. In some embodiments, any unmodified nucleotide in a sequence described herein may be modified to one of the modified nucleotides such as but not limited to those described herein. In some embodiments, a modified nucleotide in a sequence described herein may be changed to a different modified nucleotide such as but not limited to one of the modified nucleotides described herein. Modified nucleotide or modified nucleic acid encompasses modified nucleotides, bonds between nucleotides or any component of a nucleotide, and addition of one or more modified or unmodified nucleotides to one or both ends of a sequence, or addition of a cap, as described herein.

In some embodiments, a double stranded nucleic acid is provided consisting of two nucleic acid molecules selected from among SEQ ID NOs:17-18 and 34-72.

In some embodiments, a double stranded nucleic acid is provided consisting of complementary nucleic acid molecules selected from among SEQ ID NOs: 17-18 and 34-72.

In some embodiments, a double stranded nucleic acid is provided consisting of a sense strand selected from SEQ ID NOs: 17, 34, 42, 44, 45, 46, 50 and 54; and an antisense strand selected from SEQ ID NOs: 2, 18, 35, 36, 37, 38, 39, 40, 41, 43, 47, 48, 49, 51, 52, 53, 55 and 57.

In some embodiments, a double stranded nucleic acid is provided consisting of a sense strand selected from SEQ ID NOs: 17, 34, 42, 44, 45, 46, 50, 54 and 56; and an antisense strand selected from SEQ ID NOs: 2, 4, 6, 8 and 10.

In some embodiments, a double stranded nucleic acid is provided consisting of a sense strand selected from SEQ ID NOs: 1, 3, 5, 7 and 9; and an antisense strand selected from SEQ ID NOs: 18, 35, 36, 37, 38, 39, 40, 41, 43, 47, 48, 49, 51, 52, 53, 55 and 57.

In some embodiments, a double stranded nucleic acid is provided consisting of a sense strand selected from SEQ ID NOs: 17, 34, 42, 44, 45, 46, 50 and 54; and an antisense strand selected from SEQ ID NOs: 2, 4, 6, 8, 10, 18, 35, 36, 37, 38, 39, 40, 41, 43, 47, 48, 49, 51, 52, 53, 55 and 57.

In some embodiments, a double stranded nucleic acid is provided comprising a sense strand selected from SEQ ID NOs: 1, 3, 5, 7, 9, 17, 34, 42, 44, 45, 46, 50 and 54; and an antisense strand selected from SEQ ID NOs: 18, 35, 36, 37, 38, 39, 40, 41, 43, 47, 48, 49, 51, 52, 53, 55 and 57.

In some embodiments, a double-stranded nucleic acid is provided consisting of SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; or SEQ ID NO:56 and SEQ ID NO:57.

In some embodiments, a double stranded nucleic acid is provided comprising at least one nucleic acid molecule selected from among SEQ ID NOs: 17-18 or 34-57.

In some embodiments, a double stranded nucleic acid is provided comprising two nucleic acid molecules selected from among SEQ ID NOs: 17-18 or 34-57. In some embodiments, the double stranded nucleic acid comprises a sense strand and an antisense strand.

In some embodiments, each strand of the double stranded nucleic acid has no more than 52 nucleotides.

In some embodiments, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from SEQ ID NOs: 18, 35, 36, 37, 38, 39, 40, 41, 43, 47, 48, 49, 51, 52, 53, 55 and 57.

In some embodiments, a double stranded nucleic acid is provided comprising a sense strand comprising a nucleic acid molecule selected from SEQ ID NOs: 17, 34, 42, 44, 45, 46, 50, 54 and 56; and an antisense strand comprising a nucleic acid molecule selected from SEQ ID NOs: 4, 6, 8, and 10.

In some embodiments, a double stranded nucleic acid is provided comprising a sense strand comprising a nucleic acid molecule selected from SEQ ID NOs: 1, 3, 5, 7 and 9; and an antisense strand comprising a nucleic acid molecule selected from SEQ ID NO: 18, 35, 36, 37, 38, 39, 40, 41, 43, 47, 48, 49, 51, 52, 53, 55 and 57.

In some embodiments, the double-stranded nucleic acid comprises nucleic acid molecules comprising SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; or SEQ ID NO:56 and SEQ ID NO:57.

In some embodiments, a double stranded nucleic acid is provided consisting of a sense strand selected from SEQ ID NOs: 1, 17, 34, 42, 44, 45, 46, 50 and 54; and an antisense strand selected from any one of SEQ ID NOs: 58-72.

In some embodiments, a double stranded nucleic acid is provided consisting of a sense strand selected from SEQ ID NOs: 1, 3, 5 and 7; and an antisense strand selected from any one of SEQ ID NOs: 58-72.

In some embodiments, a double stranded nucleic acid is provided consisting of a sense strand selected from SEQ ID NOs: 1, 3, 5, 7, 9, 17, 34, 42, 44, 45, 46, 50 and 54; and an antisense strand selected from any one of SEQ ID NOs: 58-72.

In some embodiments, a double stranded nucleic acid is provided comprising a sense strand selected from SEQ ID NOs: 1, 3, 5, 7, 9, 17, 34, 42, 44, 45, 46, 50 and 54; and an antisense strand selected from any one of SEQ ID NOs: 58-72.

In some embodiments, a double-stranded nucleic acid is provided consisting of SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

In some embodiments, a double-stranded nucleic acid is provided consisting of SEQ ID NO:17 and any one of the following: SEQ ID NO:2, SEQ ID NO:18, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:47, SEQ ID NO:48, SEQ ID NO:49, SEQ ID NO:51, SEQ ID NO:52, SEQ ID NO:53, SEQ ID NO:55, SEQ ID NO:57, SEQ ID NO: 58, SEQ ID NO: 59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71 or SEQ ID NO:72.

In some embodiments, a double-stranded nucleic acid is provided consisting of SEQ ID NO:17 and SEQ ID NO:2; SEQ ID NO:17 and SEQ ID NO:18; SEQ ID NO:17 and SEQ ID NO:35; SEQ ID NO:17 and SEQ ID NO:36; SEQ ID NO:17 and SEQ ID NO:37; SEQ ID NO:17 and SEQ ID NO:38; SEQ ID NO:17 and SEQ ID NO:39; SEQ ID NO:17 and SEQ ID NO:40; SEQ ID NO:17 and SEQ ID NO:41; SEQ ID NO:17 and SEQ ID NO:42; SEQ ID NO:17 and SEQ ID NO:43; SEQ ID NO:17 and SEQ ID NO:44; SEQ ID NO:17 and SEQ ID NO:47; SEQ ID NO:17 and SEQ ID NO:48; SEQ ID NO:17 and SEQ ID NO:49; SEQ ID NO:17 and SEQ ID NO:51; SEQ ID NO:17 and SEQ ID NO:52; SEQ ID NO:17 and SEQ ID NO:53; SEQ ID NO:17 and SEQ ID NO:55; SEQ ID NO:17 and SEQ ID NO:57; SEQ ID NO:17 and SEQ ID NO: 58; SEQ ID NO:17 and SEQ ID NO: 59; SEQ ID NO:17 and SEQ ID NO:60; SEQ ID NO:17 and SEQ ID NO:61; SEQ ID NO:17 and SEQ ID NO:62; SEQ ID NO:17 and SEQ ID NO:63; SEQ ID NO:17 and SEQ ID NO:64; SEQ ID NO:17 and SEQ ID NO:65; SEQ ID NO:17 and SEQ ID NO:66; SEQ ID NO:17 and SEQ ID NO:67; SEQ ID NO:17 and SEQ ID NO:68; SEQ ID NO:17 and SEQ ID NO:69; SEQ ID NO:17 and SEQ ID NO:70; SEQ ID NO:17 and SEQ ID NO:71 or SEQ ID NO:17 and SEQ ID NO:72.

In some embodiments, a double-stranded nucleic acid is provided consisting of SEQ ID NO:18 and any one of SEQ ID NO:1, SEQ ID NO:17, SEQ ID NO:34, SEQ ID NO:44, SEQ ID NO:45, SEQ ID NO:46, SEQ ID NO:50, SEQ ID NO:54, or SEQ ID NO:56.

In some embodiments, a double-stranded nucleic acid is provided consisting of SEQ ID NO:18 and SEQ ID NO:1, SEQ ID NO:18 and SEQ ID NO:17; SEQ ID NO:18 and SEQ ID NO:34; SEQ ID NO:18 and SEQ ID NO:44; SEQ ID NO:18 and SEQ ID NO:45; SEQ ID NO:18 and SEQ ID NO:46; SEQ ID NO:18 and SEQ ID NO:50; SEQ ID NO:18 and SEQ ID NO:54; or SEQ ID NO:18 and SEQ ID NO:56.

In some embodiments, a double stranded nucleic acid is provided comprising at least one nucleic acid molecule selected from among SEQ ID NOs: 58-72.

In some embodiments, a double stranded nucleic acid is provided comprising two nucleic acid molecules selected from among SEQ ID NOs: 17-18 or 34-72. In some embodiments, the double stranded nucleic acid comprises a sense strand and an antisense strand.

In some embodiments, each strand of the double stranded nucleic acid has no more than 52 nucleotides.

In some embodiments, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from any one of SEQ ID NOs: 58-72.

In some embodiments, a double stranded nucleic acid is provided comprising a sense strand comprising a nucleic acid molecule selected from SEQ ID NOs: 1, 3, 5, 7 and 9; and an antisense strand comprising a nucleic acid molecule selected from any one of SEQ ID NOs:58-72.

In some embodiments, the double-stranded nucleic acid comprises nucleic acid molecules comprising SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

In some embodiments, a double-stranded nucleic acid is provided comprising SEQ ID NO:17 and comprising any one of the following: SEQ ID NO:2, SEQ ID NO:18, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:47, SEQ ID NO:48, SEQ ID NO:49, SEQ ID NO:51, SEQ ID NO:52, SEQ ID NO:53, SEQ ID NO:55, SEQ ID NO:57, SEQ ID NO: 58, SEQ ID NO: 59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71 or SEQ ID NO:72.

In some embodiments, a double-stranded nucleic acid is provided comprising SEQ ID NO:17 and SEQ ID NO:2; SEQ ID NO:17 and SEQ ID NO:18; SEQ ID NO:17 and SEQ ID NO:35; SEQ ID NO:17 and SEQ ID NO:36; SEQ ID NO:17 and SEQ ID NO:37; SEQ ID NO:17 and SEQ ID NO:38; SEQ ID NO:17 and SEQ ID NO:39; SEQ ID NO:17 and SEQ ID NO:40; SEQ ID NO:17 and SEQ ID NO:41; SEQ ID NO:17 and SEQ ID NO:42; SEQ ID NO:17 and SEQ ID NO:43; SEQ ID NO:17 and SEQ ID NO:44; SEQ ID NO:17 and SEQ ID NO:47; SEQ ID NO:17 and SEQ ID NO:48; SEQ ID NO:17 and SEQ ID NO:49; SEQ ID NO:17 and SEQ ID NO:51; SEQ ID NO:17 and SEQ ID NO:52; SEQ ID NO:17 and SEQ ID NO:53; SEQ ID NO:17 and SEQ ID NO:55; SEQ ID NO:17 and SEQ ID NO:57; SEQ ID NO:17 and SEQ ID NO: 58; SEQ ID NO:17 and SEQ ID NO: 59; SEQ ID NO:17 and SEQ ID NO:60; SEQ ID NO:17 and SEQ ID NO:61; SEQ ID NO:17 and SEQ ID NO:62; SEQ ID NO:17 and SEQ ID NO:63; SEQ ID NO:17 and SEQ ID NO:64; SEQ ID NO:17 and SEQ ID NO:65; SEQ ID NO:17 and SEQ ID NO:66; SEQ ID NO:17 and SEQ ID NO:67; SEQ ID NO:17 and SEQ ID NO:68; SEQ ID NO:17 and SEQ ID NO:69; SEQ ID NO:17 and SEQ ID NO:70; SEQ ID NO:17 and SEQ ID NO:71 or SEQ ID NO:17 and SEQ ID NO:72.

In some embodiments, a double-stranded nucleic acid is provided comprising SEQ ID NO:18 and comprising any one of SEQ ID NO:1, SEQ ID NO:17, SEQ ID NO:34, SEQ ID NO:44, SEQ ID NO:45, SEQ ID NO:46, SEQ ID NO:50, SEQ ID NO:54, or SEQ ID NO:56.

In some embodiments, a double-stranded nucleic acid is provided comprising SEQ ID NO:18 and SEQ ID NO:1, SEQ ID NO:18 and SEQ ID NO:17; SEQ ID NO:18 and SEQ ID NO:34; SEQ ID NO:18 and SEQ ID NO:44; SEQ ID NO:18 and SEQ ID NO:45; SEQ ID NO:18 and SEQ ID NO:46; SEQ ID NO:18 and SEQ ID NO:50; SEQ ID NO:18 and SEQ ID NO:54; or SEQ ID NO:18 and SEQ ID NO:56.

Any of the compositions and uses of siRNA directed to FL2 as described elsewhere herein may utilize any of the foregoing single stranded nucleic acid sequences SEQ ID NOs:58-72, or a double stranded nucleic acids comprising or consisting of any of SEQ ID NOs:58-72.

In an embodiment, the 5′ terminal residue of a strand of the siRNA is phosphorylated. In an embodiment the 5′ terminal residue of the antisense strand of the siRNA is phosphorylated. In an embodiment, the 5′ terminal residue of a strand of the siRNA is not phosphorylated. In an embodiment the 5′ terminal residue of the antisense strand of the siRNA is not phosphorylated.

In an embodiment, the siRNA comprises a double-stranded portion (duplex). In an embodiment, the siRNA is 20-25 nucleotides in length. In an embodiment the siRNA comprises a 19-21 core RNA duplex with a one or two nucleotide 3′ overhang, on, independently, either one or both strands. The siRNA can be 5′ phosphorylated, or not, and may be modified or further modified with any of the known modifications in the art to improve efficacy and/or resistance to nuclease degradation. In an embodiment the siRNA can be administered such that it is transfected into one or more cells. In an embodiment, the siRNA is 5′ phosphorylated. In some embodiments, any of the nucleic acid sequences disclosed herein may be modified or further modified with one or more nucleotide modifications as described herein.

As defined herein, the abbreviation “d(nucleotide)” refers to the deoxy-nucleotide. The abbreviation “m(nucleotide)” refers to the 2′-O-methyl nucleotide. The abbreviation “T” refers to thymidine. The abbreviation f(nucleotide) refers to the 2′-fluorodeoxy nucleotide. The abbreviation “(Phos)” refers to a phosphodiester cap. A capital letter residue refers to an RNA residue. The abbreviation “l(nucleotide)” refers to a locked nucleotide. A locked nucleotide has an extra bridge connecting the 2′ oxygen and 4′ carbon. The abbreviation “(s)” refers to phosphorothioate, i.e., a phosphorothioate bond between the adjacent nucleotides or modified nucleotides. Otherwise, the abbreviations for nucleotides and ribonucleotides have the meaning known in the art.

The abbreviations of the modifications of nucleotides described herein are as follows. mU refers to 2′-O-methyl-uridine. mA refers to 2′-O-methyl-adenosine. mC refers to 2′-O-methyl-cytidine. mG refers to 2′-O-methyl-guanosine. fA refers to 2′-fluoro-adenosine. fC refers to 2′-fluoro-cytidine. fG refers to 2′-fluoro-guanosine. fU refers to 2′-fluoro-uridine. dC refers to deoxycytidine. dG refers to deoxyguanosine. dA refers to deoxyadenosine. A refers to adenine. The abbreviations for the bases of unmodified nucleotides include A refers to adenine; U refers to uracil; G refers to guanine; C refers to cytosine; T refers to thymine. lA (lower case L A) refers to a locked adenosine. lU refers to a locked uridine. lG refers to a locked guanosine. lC refers to a locked cytidine.

In some embodiments, any of the nucleic acid sequences disclosed herein may be modified or further modified with one or more modifications or additional modifications as described herein. In addition to the modifications described above present in the sequences listed herein, and may be further included in any of the nucleic acids at other positions not modified or replacements for those already modified, other nucleic acid modifications are fully encompassed herein. Such other modifications include 2′-O-methyl thymidine, 2′-fluoro thymidine, and deoxyuridine.

It should be noted that the abbreviations herein of the unmodified and modified nucleic acid abbreviations may refer to the nucleic acid base, the nucleoside (i.e., the base and the sugar), or the nucleotide (the nucleoside and the phosphate group). One of skill in the art will recognize the unmodified or modified nucleic acid components therefrom.

A locked nucleic acid (LNA), often referred to as inaccessible RNA, is a modified RNA nucleotide in which the ribose moiety is modified with an extra bridge connecting the 2′ oxygen and 4′ carbon. In some embodiments, a nucleic acid comprises a locked adenosine. In some embodiments a nucleic acid comprises a locked cytosine. In some embodiments a nucleic acid comprises a locked guanosine. In some embodiments a nucleic acid comprises a locked uridine. In some embodiments a nucleic acid comprises a locked thymidine.

In an embodiment, the 5′ terminal residue of a strand of the siRNA is phosphorylated. In an embodiment the 5′ terminal residue of the antisense strand of the siRNA is phosphorylated.

In some embodiments, a single strand component of a siRNA of the disclosure is from 14 to 50 nucleotides in length. In another embodiment, a single strand component of a siRNA of the disclosure is 14, 15, 16, 17, 18, 19, 20, 21, 22, 23, 24, 25, 26, 27, or 28 nucleotides in length. In yet another embodiment, a single strand component of a siRNA of the disclosure is 21 nucleotides in length. In yet another embodiment, a single strand component of a siRNA of the disclosure is 22 nucleotides in length. In yet another embodiment, a single strand component of a siRNA of the disclosure is 23 nucleotides in length. In some embodiments, a siRNA of the disclosure is from 28 to 56 nucleotides in length. In another embodiment, a siRNA of the disclosure is 40, 41, 42, 43, 44, 45, 46, 47, 48, 49, 50, 51, or 52 nucleotides in length.

In another embodiment, an siRNA of the disclosure comprises at least one 2′-sugar modification. In another embodiment, an siRNA of the disclosure comprises at least one nucleic acid base modification. In another embodiment, an siRNA of the disclosure comprises at least one phosphate backbone modification. As used herein, “at least one” means one or more.

The NCBI reference sequence: NM 001013690.4 (SEQ ID NO:19), to the nucleic acid encoding human fidgetin-like 2, is:

| (SEQ ID NO: 19) | |
|---|---|
| 1 | agtgagctat ggggacacta ctgcactgta gcctgggcaa cagagcaaga ccttgtctca |
| 61 | aaaatgtata tatattttgg gctttttttc ctaaaacggg aactacaaca gcatatttgc |
| 121 | gagctgatga gagtgaccca gcagagaggg aaatggatca gctctgttga agatgcactg |
| 181 | gacaccagaa cacgcccagc ccctcaacca gtggccagag cagcacctgg acgtctcctc |
| 241 | caccaccccg tcgccggccc acaagttgga gttgccccct gggggtcgcc aacgctgcca |
| 301 | ctacgcttgg gcacacgacg acatctcagc cctcactgcc tccaacctcc taaagcgcta |
| 361 | tgcagagaag tactctgggg tcttggattc tccctacgag cgtccggccc tgggcgggta |
| 421 | cagcgacgcc tccttcctca acggcgccaa aggggatccc gagccctggc cagggccgga |
| 481 | gccaccctac cccttggcct cactccacga aggcctocca ggaaccaaat cgggcggtgg |
| 541 | cggcggttcc ggggccctgg ggggctcccc agttttagcc gggaacctcc ctgaacccct |
| 601 | ctacgccggc aatgcgtgcg ggggcccatc ggcggcgccc gagtacgcgg ccggctacgg |
| 661 | cggggggtac ctggcgccgg gttactgcgc gcagacgggc gccgcgctgc ccccgccgcc |
| 721 | cccggccgcg ctcctgcagc ccccaccgcc tccggggtac gggccctcag cgccgctgta |
| 781 | caactatccc gcagggggct acgcagcgca gcccggctat ggcgcgctcc cgccgccccc |
| 841 | aggcccaccc ccggccccct acctgacccc gggcctgccc gcgcccacgc ccctgcccgc |
| 901 | gccggcaccg cccaccgcct atggcttccc cacggccgcg ccgggtgccg aatccgggct |
| 961 | gtcgctgaag cgcaaggccg ccgacgaggg gcccgagggc cgctaccgca agtacgcgta |
| 1021 | cgagcccgcc aaggcccccg tggctgacgg agcctcctac cccgccgcgg acaacggcga |
| 1081 | atgtcggggc aacgggttcc gggccaagcc gccaggagcc gcggaggagg cgtcgggcaa |
| 1141 | gtacggtggc ggcgtccccc tcaaggtcct gggctccccc gtctacggcc cgcaactgga |
| 1201 | gccctttgaa aagttcccgg agcgggcccc ggctcctcgt ggggggttcg ccgtgccgtc |
| 1261 | gggggagact cccaaaggcg tggaccctgg ggccctggag ctggtgacga gcaagatggt |
| 1321 | ggactgcggg cccccggtgc agtgggcgga tgtggcgggc cagggcgcgc tcaaggcggc |
| 1381 | gctggaggag gagctggtgt ggcccctgct caggccgccc gcctacccgg gcagcctgcg |
| 1441 | cccgccgcgg accgtcctgc tctttgggcc gcggggcgcg ggcaaagcgc tgctgggccg |
| 1501 | ctgcctcgcc acgcagctgg gcgccacgct gttgcgcctg cgcggcgcga ccctggctgc |
| 1561 | gcccggcgcc gccgagggcg cgcgcctcct ccaggccgcc ttcgcggccg cgcgctgccg |
| 1621 | cccaccctcc gtactcctca tcagcgagct agaggcgctg ctccccgccc gggacgacgg |
| 1681 | cgcggcggca gggggcgcgc tgcaggtgcc gctcctggcc tgcctggacg ggggctgcgg |
| 1741 | cgcgggggct gacggcgtgc tggttgtggg caccacctcg cggcccgcgg ctctggacga |
| 1801 | ggcgacccgc cggcgcttct ctctccgctt ctacgtggcg ctgcccgaca gcccggcccg |
| 1861 | cgggcagatc ctgcagcggg cgctggccca gcagggctgc gcgctcagtg agcgggaact |
| 1921 | ggcggcgctg gtgcagggca cgcagggctt ctctgggggc gagctggggc agctgtgcca |
| 1981 | gcaggcggcg gccggggcgg gcctcccggg gctgcagcgc cccctctcct acaaggacct |
| 2041 | ggaggcggcg ctggccaagg tgggccctag ggcctctgcc aaggaactgg actcgttcgt |
| 2101 | ggagtgggac aaaatgtacg gctccggaca ctgacggcgc gcgggggagg ccgcgggagc |
| 2161 | cgcagtccct ccgtccccgc cgcctccgcg tgggagggat gtcactgact aaacccggct |
| 2221 | ggcaggggct ggagtggtga atgtgggatc ggggacagga ggggtctgcc ggtggatatt |
| 2281 | ttttttttcg tgggaaggaa aatgcttctg ccaggcagat gccatatgcg ccgtgtactc |
| 2341 | aggtttttcc tatttattgt ggactggaag ctcgccatct ccgcccggca gaccgggcag |
| 2401 | atccggcatg ggctggcacc cggggcctta agaactcctg ctctcttgcc acaacgcttt |
| 2461 | tgtctcctcg ctatctgaat ggcaccctcc ttctccctca ctctctccat cccattctct |
| 2521 | gcattctctt ggttttctct cccttttgct ttgtcgctga cacccctgcc caccccatgc |
| 2581 | tggccctgtt tctctcctgc ccctccctcc ccagctctcc atccctcacc ctctgtgctt |
| 2641 | ctgtctccat ccctggctct ccagcgtccc tggccttttg gtccctgagc tttaatgcct |
| 2701 | ttccctgcct tctgttctta tttggactgc agtggccctt tgcaggagct ctggaggccc |
| 2761 | aggggctgag gaggagggtt acccctctac ccatctgaaa cctagggtct agggggatca |
| 2821 | aggaaaaaaa gtccccaaag aaggggaatt ttttgtttgt ttttgagggg agatcccaga |
| 2881 | aatgtagctt gtttcatatt ttagtcttct tatttttgta aaatgtgtag aatttgctgt |
| 2941 | ttttcttttt cttttgacaa ctcaggaaga aactgacctc agaaagaatg ttagactttg |
| 3001 | gctgctctcc tgtgtgcccc tcacacctgc cccctccccc ccactccatc caggggacca |
| 3061 | aattctccca gacactcaaa aaatgagact tacggggaag gggagaggaa gacccagagg |
| 3121 | cctcagtgaa accccagcta ttcctggtca gaagcagaat gtattcctaa gggcttcctc |
| 3181 | cccagggccg aggcctaggc atgaatgtgg ggagtgggct gtggggtttg agagaaggga |
| 3241 | ggccttattc ctctcctgct gctccccacc ccctgcccca cccaacccct ccgctgagtg |
| 3301 | ttttctgtga agggctatcc agagttagga tgcccttgcc caattccttc ctgagaccca |
| 3361 | gaaggtaggg tgggagggcc caaatgggaa ggtgacctaa gcagaaagtc tccagaaagg |
| 3421 | tcatgtcccc tggccctgcc ttggcagagg tccccagtga cttatgctag gaggattcca |
| 3481 | tctgggtaga cagtctggcc acaaaatcag ctactggacc tcagccatct ctgctggagg |
| 3541 | ctctgaggag gagtgagcat ccctcacttg tgggggctct gtgaggaaat gtgccttccc |
| 3601 | cattcccccg gagtcctagg tctggagctc cagggctggg agagggtgag ggagatgggc |
| 3661 | aggggtgttt tctctgacct tgggggctta gtctcagtcc tgcctgaact ttccactagg |
| 3721 | cttggaaccc ttccaagaac catatttctc tccttcccac caattttccc ttgatgaggc |
| 3781 | tttagcagtt tgctcccacc acccccagcc catttcacaa ctctgatctt agtccaaagc |
| 3841 | aggggacacg cccccccacc accacttttt ctctctccca tctcagcctc ctgtgcagtt |
| 3901 | ccttgcctgc ccgtgcattt cctagagtct actgcctccc ccctggctgg gagggtgtct |
| 3961 | gggggggatc tttcaggggc cctggcaccc agggcctgtg ctggcctagg agtgctgacc |
| 4021 | agaaggctgc tctgttcccc cccacccccg ttgctttctg gccccctctt tggagccagc |
| 4081 | cacccacagg gctttggtgc ctcagaagca gtgggctgcc gggtcacagc cgcaggctgc |
| 4141 | aaaagaccct cggagggagc atggagtgag gggttctctc tcaggtgtgt atgtattggg |
| 4201 | gggtgggggt gggtggaggg tgtcagggaa gttggggtgg gatcccagcc ttcccttcaa |
| 4261 | gaggcaggga gctctgggag gtggagtccc caccgctttc tctactaggc tcctcctgtt |
| 4321 | ccccaggctt ggggagcttt gcacaaggag actgccccca gcctagtggc acctacctca |
| 4381 | tgggctctgg ggcaggtagg ggaagggcca gtccagctct ggtaatgctg gggggaggca |
| 4441 | taccaaagaa tccaggggca gggagtgggg agggtgactt ccgagctggc ctctcccctt |
| 4501 | cctctaccca gactggggct gggatcctct cctcccgctg taaccattto tacctcattt |
| 4561 | tgctgcgtgt tgtacatgga cgtatttatc tcctgtctga cgatgctctg cagttgtggt |
| 4621 | ctgtctacct cagaagagac tgtattttaa aagaaagtat tacacagtat taaagcgatg |
| 4681 | acatgtggtt tgcaaaaaaa aaaaaaaaaa a |

which encodes:

(SEQ ID NO: 2)
MHWTPEHAQPLNQWPEQHLDVSSTTPSPAHKLELPPGGRQRCHYAWAHD
DISALTASNLLKRYAEKYSGVLDSPYERPALGGYSDASFLNGAKGDPEP
WPGPEPPYPLASLHEGLPGTKSGGGGGSGALGGSPVLAGNLPEPLYAGN
ACGGPSAAPEYAAGYGGGYLAPGYCAQTGAALPPPPPAALLQPPPPPGY
GPSAPLYNYPAGGYAAQPGYGALPPPPGPPPAPYLTPGLPAPTPLPAPA
PPTAYGFPTAAPGAESGLSLKRKAADEGPEGRYRKYAYEPAKAPVADGA
SYPAADNGECRGNGFRAKPPGAAEEASGKYGGGVPLKVLGSPVYGPQLE
PFEKFPERAPAPRGGFAVPSGETPKGVDPGALELVTSKMVDCGPPVQWA
DVAGQGALKAALEEELVWPLLRPPAYPGSLRPPRTVLLFGPRGAGKALL
GRCLATQLGATLLRLRGATLAAPGAAEGARLLQAAFAAARCRPPSVLLI
SELEALLPARDDGAAAGGALQVPLLACLDGGCGAGADGVLVVGTTSRPA
ALDEATRRRFSLRFYVALPDSPARGQILQRALAQQGCALSERELAALVQ
GTQGFSGGELGQLCQQAAAGAGLPGLQRPLSYKDLEAALAKVGPRASAK
ELDSFVEWDKMYGSGH (human fidgetin-like 20).

The nucleic acid sequences are disclosed herein.

| RNA sequence | Sequence ID |
|---|---|
| UUACACAGUAUUAAAGCGAUU (sense) | SEQ ID NO: 1 |
| UCGCUUUAAUACUGUGUAAUU (antisense) | SEQ ID NO: 2 |
| CAUCUGAAACCUAGGGUCUUU (sense) | SEQ ID NO: 3 |
| AGACCCUAGGUUUCAGAUGUU (antisense) | SEQ ID NO: 4 |
| GUGACUUAUGCUAGGAGGAUU (sense) | SEQ ID NO: 5 |
| UCCUCCUAGCAUAAGUCACUU (antisense) | SEQ ID NO: 6 |
| GGUCAGAAGCAGAAUGUAUUU (sense) | SEQ ID NO: 7 |
| AUACAUUCUGCUUCUGACCUU (antisense) | SEQ ID NO: 8 |
| CGCCGGCCCACAAGUUGGAdTdT (sense) | SEQ ID NO: 9 |
| UCCAACUUGUGGGCCGGCGdTdT (antisense) | SEQ ID NO: 10 |
| fUfUmA fCmAfC AGU AUU AAA GCG ATT (sense) | SEQ ID NO: 17 |
| (Phos) U CGC UUU AAU ACU G UG UAA TT (antisense) | SEQ ID NO: 18 |
| 5′-UUACACAGUAUUAAAGCGATT-3′ (sense) | SEQ ID NO: 34 |
| (Phos) 5′-mUmCGCUUUAAUACUGUGUAATT-3′ (antisense) | SEQ ID NO: 35 |
| (Phos) 5′-mU(s)mC(s)GCUUUAAUACUGUGUAATT-3′ (antisense) | SEQ ID NO: 36 |
| (Phos) 5′-fUfCGCUUUAAUACUGUGUAATT-3′ (antisense) | SEQ ID NO: 37 |
| (Phos) 5′-fU(s)fC(s)GCUUUAAUACUGUGUAATT-3′(antisense) | SEQ ID NO: 38 |
| (Phos) 5′-mU(s)mC(s)GCUUUAAUAmCfUmGfUmGfUmAmATT-3′ (antisense) | SEQ ID NO: 39 |
| (Phos) 5′-U(s)CGCUUUAAUACUGUGUAATT-3′ (antisense) | SEQ ID NO: 40 |
| (Phos) 5′-mUfCmGfCmUfUmUAAfUmAfCmUGmUmGfUmAmATT (antisense) | SEQ ID NO: 41 |
| 5′ mUmUmAmCmAmCmAmGmUmAmUmUmAmAmAmGmCmGmAmUmU-3′ (antisense) | SEQ ID NO: 42 |
| (Phos) 5′-mUmCmGmCmUmUmUmAmAmUmAmCmUmGmUmGmUmAmAmUmU-3′ (antisense) | SEQ ID NO: 43 |
| 5′ mUmUmAmCmAmCmAmGmUmAmUmUmAmAmAmGdCdGdATT-3′ (sense) | SEQ ID NO: 44 |
| 5′ mUmUmAmCmAmCmAmGmUmAmUmUmAmAmAmGdCmGmATT-3′ (sense) | SEQ ID NO: 45 |
| 5′ UUACACAGUAUUAAAGCGA-3′ (sense) | SEQ ID NO: 46 |
| (Phos) 5′-U(s)CGCUUUAAUACUGUGUAATT-3′ (antisense) | SEQ ID NO: 47 |
| (Phos) 5′-UCGCUUUAAUACUGUGUAATT-3′(antisense) | SEQ ID NO: 48 |
| (Phos) 5′-U(s)C(s)GCUUUAAUACUGUGUAATT-3′(antisense) | SEQ ID NO: 49 |
| 5′-mUmUACACAGUAUUAAAGCGA-3′(sense) | SEQ ID NO: 50 |
| (Phos) 5′-U(s)CGCUUUAAUACUGUGUmAmATT-3′(antisense) | SEQ ID NO: 51 |
| (Phos) 5′-UCGCUUUAAUACUGUGUAATT-3′ (antisense) | SEQ ID NO: 52 |
| (Phos) 5′-U(s)C(s)GCUUUAAUACUGUGUAA T(s)T-3′ (antisense) | SEQ ID NO: 53 |
| 5′ lUlUlAlClACAGUAUUAAAGCGATT-3′ (sense) | SEQ ID NO: 54 |
| (Phos) 5′-UCGCUUUAAUACUGlUlGlUlAlA TT-3′ (antisense) | SEQ ID NO: 55 |
| 5′ fUfUlAfClACAGUAUUAAAGCGA-3′ (sense) | SEQ ID NO: 56 |
| (Phos) 5′-mU(s)mCmGCUUUAAUACUGUGUAATT-3′ (antisense) | SEQ ID NO: 57 |
| 5′-fUfCGCUUUAAUACUGUGUAATT-3′ (antisense) | SEQ ID NO: 58 |
| 5′-fU(s)fC(s)GCUUUAAUACUGUGUAATT-3′(antisense) | SEQ ID NO: 59 |
| 5′-mU(s)mC(s)GCUUUAAUAmCfUmGfUmGfUmAmATT-3′ (antisense) | SEQ ID NO: 60 |
| 5′-U(s)CGCUUUAAUACUGUGUAATT-3′ (antisense) | SEQ ID NO: 61 |
| 5′-mUfCmGfCmUfUmUAAfUmAfCmUGmUmGfUmAmATT (antisense) | SEQ ID NO: 62 |
| 5′ mUmCmGmCmUmUmUmAmAmUmAmCmUmGmUmGmUmAmAmUmU-3′ (antisense) | SEQ ID NO: 63 |
| 5′-U(s)CGCUUUAAUACUGUGUAATT-3′ (antisense) | SEQ ID NO: 64 |
| 5′-UCGCUUUAAUACUGUGUAATT-3′(antisense) | SEQ ID NO: 65 |
| 5′-U(s)C(s)GCUUUAAUACUGUGUAATT-3′(antisense) | SEQ ID NO: 66 |
| 5′-U(s)CGCUUUAAUACUGUGUmAmATT-3′(antisense) | SEQ ID NO: 67 |
| 5′-UCGCUUUAAUACUGUGUAATT-3′ (antisense) | SEQ ID NO: 68 |
| 5′-U(s)C(s)GCUUUAAUACUGUGUAA T(s)T-3′ (antisense) | SEQ ID NO: 69 |
| 5′-UCGCUUUAAUACUGlUlGlUlAlA TT-3′ (antisense) | SEQ ID NO: 70 |
| 5′-mU(s)mCmGCUUUAAUACUGUGUAATT-3′ (antisense) | SEQ ID NO: 71 |
| 5′-U CGC UUU AAU ACU G UG UAA TT (antisense) | SEQ ID NO: 72 |

wherein d(nucleotide) = deoxy-(nucleotide), m(nucleotide) = 2′-O-methyl nucleotide, T =
thymidine, f(nucleotide) = 2′-fluorodeoxy nucleotide, (Phos) = phosphodiester cap; capital
letter nucleotide = RNA nucleotide, l(nucleotide) = a locked nucleotide, and (s) =
phosphorothioate.

Thus, for example dT represents deoxythymidine, dC represents deoxycytidine, fC represents 2′-fluorodeoxy cytidine ribonucleic acid, fU represents 2′-fluorodeoxy uracil ribonucleic acid, mA represents 2′-O-methyl adenosine ribonucleic acid, mU represents 2′-O-methyl uracil ribonucleic acid, mC represents 2′-O-methyl cytosine ribonucleic acid, and mG represents 2′-O-methyl guanosine ribonucleic acid.

The disclosure embraces modifications of the nucleic acids sequences disclosed herein that are useful for treatment of a non-human animals (e.g., non-human primate, non-human mammal). Such modifications of the nucleic acids disclosed herein comprise siRNAs directed to the orthologue of fidgetin-like 2 in the particular species.

### Pharmaceutical Compositions

In some embodiments, a formulation, pharmaceutical composition, or delivery system of any of the nucleic acids described herein is provided. In some embodiments, the formulation, pharmaceutical composition, or delivery system comprises a nucleic acid consisting of or comprising any of those nucleic acids described herein, such as single stranded and double stranded or a duplex. In some embodiments, the formulation comprises one or more nucleic acids selected from among SEQ ID NO:17-18 and 34-72, or a duplex or double-stranded nucleic acid comprising a nucleic acid consisting of two nucleic acid molecules selected from among SEQ ID NO: 17-18 and 34-72. In some embodiments, the formulation, pharmaceutical composition, or delivery system comprises a nucleic acid comprising a sequence selected from SEQ ID NO: 17-18 or 34-72, or a duplex or double-stranded nucleic acid comprising SEQ ID NO: 17-18 or 34-72. In any of the following descriptions of formulations, pharmaceutical compositions, or delivery systems, any of the foregoing nucleic acids or those described elsewhere herein are embodied, and may be referred to as an inhibitor of fidgetin-like 2. In some embodiments, compounds of the disclosure inhibit activity of fidgetin-like 2 (nucleic acid sequence SEQ ID NO1; protein sequence SEQ ID NO:20.

In an embodiment of the disclosure the inhibitor of fidgetin-like 2 is provided by a subcutaneous implant or depot medicament system for the pulsatile delivery of the inhibitor to a wound or to a site where a wound is expected to be formed, for example, after surgery, to promote wound healing. The inhibitor can be provided, for example, in a therapeutically effective amount to each centimeter of a wound margin or each centimeter of a site at which a wound is expected to be formed.

A medicament in accordance with this aspect of the disclosure may be formulated in any appropriate carrier, vehicle, diluent, excipient or other delivery system. Suitable carriers are pharmaceutically acceptable carriers, preferably those consistent with administration topically or administration by injection.

It will be appreciated that, while the inhibitor of fidgetin-like 2 may be administered by the same route and in the same form in each incidence of treatment, different incidences of treatment may provide the inhibitor of fidgetin-like 2 by different medicaments and/or different routes of administration. In embodiments of the disclosure the initial incidence of treatment may provide the inhibitor of fidgetin-like 2 by means of an injection, such as an intradermal injection, while the second (and any subsequent) incidences of treatment may involve provision of the inhibitor of fidgetin-like 2 by alternative routes, such as topical formulations, or vice versa. In an embodiment, multiple administrations of the inhibitor of fidgetin-like 2 may be affected by the same means or route.

In some embodiments the inhibitor of fidgetin-like 2 is provided in a pharmaceutical composition comprising saline. In some embodiments the pharmaceutical composition is normal saline or phosphate-buffered saline.

In some embodiments the inhibitor of fidgetin-like 2 is provided in a pharmaceutical composition comprising a sugar. In some embodiments, the pharmaceutical composition is a glucose solution.

In some embodiments the inhibitor of fidgetin-like 2 is provided in a pharmaceutical composition comprising a polypeptide. In some embodiments the polypeptide is a cationic polypeptide. In some embodiments, the cationic polypeptide is a histidine-lysine copolypeptide.

In some embodiments the inhibitor of fidgetin-like 2 is provided in a pharmaceutical composition comprising a polymer. In some embodiments, the hydrophilic polymer is polyethylene glycol (PEG). In some embodiments, the polymer is a hydrophilic polymer grafted polymer, a non-natural cationic polymer, a cationic polyacetal, a hydrophilic polymer grafted polyacetal, a ligand functionalized cationic polymer, or a ligand functionalized-hydrophilic polymer grafted polymer. In some embodiments the hydrophilic polymer is polyethylene glycol (PEG).

In some embodiments, the pharmaceutical composition comprises a polycationic binding agent.

In some embodiment, the pharmaceutical composition comprises a nucleic acid delivery vehicle.

In some embodiments, the pharmaceutical composition comprises a cationic polymer-nucleic acid complex.

In some embodiments the inhibitor of fidgetin-like 2 is provided in a pharmaceutical composition comprising a lipid. In some embodiments the lipid is a cationic lipid.

In some embodiments the inhibitor of fidgetin-like 2 is provided in a pharmaceutical composition comprising a cream.

In some embodiments the inhibitor of fidgetin-like 2 is provided in a pharmaceutical composition comprising an eye drop.

In some embodiments the inhibitor of fidgetin-like 2 is provided in a pharmaceutical composition comprising a gel.

In some embodiments the inhibitor of fidgetin-like 2 is provided in a pharmaceutical composition comprising a micelle material.

In some embodiments the inhibitor of fidgetin-like 2 is provided in a pharmaceutical composition comprising a wafer. In some embodiments, a wafer comprises collagen, chondroitin sulfate, polyvinylpyrrolidone and polyethylene glycol 400. One non-limiting example of a wafer is described in an example herein.

In some embodiments, the inhibitor of fidgetin-like 2 is provided in or associated with a collagen particle. In some embodiments the collagen particle is a microparticle. In some embodiments the collagen particle is in a surfactant polymer dressing such as but not limited to PluroGel®.

In some embodiments the inhibitor of fidgetin-like 2 is provided in a pharmaceutical composition comprising a microemulsion of nanoparticles. One non-limiting example of a microemulsion is described in an example herein.

In some embodiments the inhibitor of fidgetin-like 2 is provided in a pharmaceutical composition comprising a liposome. In some embodiments, the liposome is a ligand functionalized liposome. In some embodiments, the liposome is further functionalized with at least one 2′ sugar modification.

In some embodiments the inhibitor of fidgetin-like 2 is provided in a pharmaceutical composition comprising a nanoparticle. In an embodiment, the inhibitor of fidgetin-like 2 is encapsulated in a nanoparticle. In an embodiment the nanoparticle is a liposomal nanoparticle.

In one non-limiting example of nanoparticles are prepared as follows. Five hundred μl of tetramethyl orthosilicate (TMOS) was hydrolyzed in the presence of 100 μl of 1 mM HCl by sonication on ice for about 15 min, until a single phase formed. The hydrolyzed TMOS (100 μl) was added to 900 μl of 20 μM of siRNA solution containing 10 mM phosphate, pH 7.4. A gel was formed within 10 minutes. The gel was frozen at −80° C. for 15 minutes and lyophilized.

In some exemplary, but non-limiting, embodiments, the nanoparticle comprises a poly(lactic-co-glycolic acid) (PLGA, PLG), a copolymer, produced using methods known in the art. In some embodiments, the nanoparticle is sized between 1-100 nm. In some embodiments, the nanoparticle is biocompatible and/or biodegradable. This addition may in certain embodiments enhance purification of microparticles or nanoparticles using methods well known in the art.

In a non-limiting embodiment, the inhibitor of fidgetin-like 2 is provided in a bulk-eroding system such as polylactic acid and glycolic acid (PLGA) copolymer-based microspheres or microcapsules systems containing the inhibitor of fidgetin-like 2. In an embodiment, blends of PLGA:ethylcellulose systems may be used as an appropriate carrier. A further medicament in accordance with this aspect of the disclosure may be formulated in a surface-eroding system wherein the inhibitor of fidgetin-like 2 is embedded in an erodible matrix such as the poly(ortho) ester and polyanhydride matrices wherein the hydrolysis of the polymer is rapid.

In some embodiments, the inhibitor of fidgetin-like 2 may also be formulated by combining a pulsatile delivery system as described above and an immediate release system such as a lyophilized injectable composition described above.

The inhibitor of fidgetin-like 2 may be used in a composition with additives. Examples of suitable additives are sodium alginate, as a gelatinizing agent for preparing a suitable base, or cellulose derivatives, such as guar or xanthan gum, inorganic gelatinizing agents, such as aluminum hydroxide or bentonites (termed thixotropic gel-formers), polyacrylic acid derivatives, such as Carbopol®, polyvinylpyrrolidone, microcrystalline cellulose and carboxymethylcellulose. Amphiphilic low molecular weight and higher molecular weight compounds, and also phospholipids, are also suitable. The gels can be present either as water-based hydrogels or as hydrophobic organogels, for example based on mixtures of low and high molecular weight paraffin hydrocarbons and vaseline. The hydrophilic organogels can be prepared, for example, on the basis of high molecular weight polyethylene glycols. These gelatinous forms are washable. Hydrophobic organogels are also suitable. Hydrophobic additives, such as petroleum jelly, wax, oleyl alcohol, propylene glycol monostearate and/or propylene glycol monopalmitostearate, in particular isopropyl myristate can be included. In an embodiment the inhibitor is in a composition comprising one or more dyes, for example yellow and/or red iron oxide and/or titanium dioxide for the purpose of matching as regards color.

Compositions may be in any suitable form including gels, lotions, balms, pastes, sprays, powders, bandages, wound dressing, emulsions, creams and ointments of the mixed-phase or amphiphilic emulsion systems (oil/water-water/oil mixed phase), liposomes and transfersomes or plasters/band aid-type coverings. Emulsifiers which can be employed in compositions comprising the inhibitor of fidgetin-like 2 include anionic, cationic or neutral surfactants, for example alkali metal soaps, metal soaps, amine soaps, sulphurated and sulphonated compounds, invert soaps, higher fatty alcohols, partial fatty acid esters of sorbitan and polyoxyethylene sorbitan, e.g. lanette types, wool wax, lanolin or other synthetic products for preparing the oil/water and/or water/oil emulsions.

Compositions comprising the inhibitor of fidgetin-like 2 can also comprise vaseline, natural or synthetic waxes, fatty acids, fatty alcohols, fatty acid esters, for example as monoglycerides, diglycerides or triglycerides, paraffin oil or vegetable oils, hydrogenated castor oil or coconut oil, hog fat, synthetic fats (for example based on caprylic acid, capric acid, lauric acid or stearic acid, such as Softisan®), or triglyceride mixtures, such as Miglyol®, can be used as lipids, in the form of fatty and/or oleaginous and/or waxy components for preparing the ointments, creams or emulsions of the compositions comprising the inhibitor of fidgetin-like 2 used in the methods described herein.

In some embodiments, the pharmaceutical composition comprises an osmotically active acid or alkaline solution, for example hydrochloric acid, citric acid, sodium hydroxide solution, potassium hydroxide solution, sodium hydrogen carbonate, may also be ingredients of the compositions and, in addition, buffer systems, such as citrate, phosphate, tris buffer or triethanolamine, for adjusting the pH. It is possible to add preservatives as well, such as methyl benzoate or propyl benzoate (parabens) or sorbic acid, for increasing the stability.

Pastes, powders and solutions are additional forms of compositions comprising the inhibitor of fidgetin-like 2 which can be applied topically. As consistency-imparting bases, the pastes frequently contain hydrophobic and hydrophilic auxiliary substances, preferably, however, hydrophobic auxiliary substances containing a very high proportion of solids. In order to increase dispersity, and also flowability and slipperiness, and also to prevent agglomerates, the powders or topically applicable powders can, for example, contain starch species, such as wheat or rice starch, flame-dispersed silicon dioxide or siliceous earth, which also serve as diluent.

In an embodiment, the compositions comprise further active ingredients suitable for protecting or aiding in healing of the wound, for example one or more antibiotics, antiseptics, vitamins, anesthetics, antihistamines, anti-inflammatory agents, moisturizers, penetration-enhancing agents and/or anti-irritants.

Preferably the inhibitor of fidgetin-like 2 is biomembrane-permeable or is conjugated or otherwise attached to a moiety which renders the inhibitor biomembrane-permeable.

In some embodiments, the carrier further comprises a targeting ligand. In some embodiments the targeting ligand is a protein. In some embodiments, the targeting ligand binds an epithelial cell, a vascular endothelial cell, a vascular smooth muscle cell, a myocardial (heart) cell or a passenger leukocyte cell resident in cutaneous tissue at a time of wound healing.

In some embodiments, the carrier comprises: (a) a histidine-lysine co-polymer; (b) a hydrophilic polymer comprising PEG; and, optionally, (c) a targeting ligand.

In an embodiment, the composition may further comprise one or more additional nucleic acid molecules that induce RNA interference and decrease the expression of a gene of interest. In an embodiment, the one or more additional nucleic acid molecules decrease the expression of a gene selected from the group consisting of fidgetin and fidgetin-like 2.

### Methods of Use

In some embodiments, methods of use of any of the nucleic acids described herein and their pharmaceutical compositions are provided. In some embodiments, methods of use are provided using a nucleic acid consisting of SEQ ID NO:17-18 or 34-72, or a duplex or double-stranded nucleic acid comprising nucleic acids consisting of from SEQ ID NO:17-18 or 34-72. In some embodiments, the methods of use are provided using a nucleic acid comprising a sequence selected from SEQ ID NO:17-18 or 34-72, or a duplex or double-stranded nucleic acid comprising two sequences from among SEQ ID NO:17-18 or 34-72. In some embodiments, methods of use are provided using a nucleic acid consisting of any one of SEQ ID NO:17-18 or 34-72, and any complementary nucleic acid disclosed herein. In some embodiments, methods of use are provided using a duplex or double-stranded nucleic acid comprising a nucleic acid comprising any of from SEQ ID NO:17-18 or 34-72, and any complementary nucleic acid disclosed herein. In some embodiments, modifications or additional modifications to the nucleic acid such as but not limited to those described herein is embraced herein. In any of the following descriptions of methods of use, any of the foregoing nucleic acids or those described elsewhere herein are embodied. The term “an inhibitor of fidgetin-like 2” is meant to encompass any of the nucleic acid sequences described herein and modifications thereof. In some embodiments each individual strand within the double-stranded nucleic acid is no longer than 52 nucleotides. The disclosure embraces method of use for treatment of a non-human animals (e.g., non-human primate, non-human mammal) comprising modifications of the nucleic acids sequences disclosed herein. Such modifications of the nucleic acids disclosed herein comprise siRNAs directed to the orthologue of fidgetin-like 2 in the particular species. In some embodiments, the nucleic acids and siRNA disclosed herein are cross-reactive and therefore useful in at least one other species.

The following descriptions provide non-limiting guidance as to the various wounds, injuries and diseases, among other conditions, that the compounds and compositions of the disclosure may benefit. The following descriptions are categorized by bodily system or site, with the recognition that such categorizations are for convenience only, and that certain aspects are shared among the categories and such categorization is not intended to be limiting to the particular conditions, diseases, wounds or injuries of any particular bodily system or site.

### Treatment of Skin

A method of treating a wound in a subject is provided comprising administering to the subject an amount of an inhibitor of fidgetin-like 2 effective to treat the wound.

In an embodiment, the amount of inhibitor of fidgetin-like 2 is effective to accelerate wound healing.

In an embodiment, the wound is an epidermal wound. In an embodiment, the wound is a skin wound. Non-limiting examples of specific wounds in which healing may be promoted using the medicaments and methods of the disclosure include, but are not limited to, the results of sun damage such as wrinkles, non-responsive skin after a facelift, lasabrasion, aged or sun-damaged skin, skin liver spots, birthmark, wart, enlarged oil glands, port wine stains, hemangiomas, telangiectasias, or to change the appearance of skin complexion. In an embodiment of the methods, the birthmark is a linear epidermal nevus. In some embodiments, the method is directed to enhancing skin health recovery from a skin procedure comprising laser application to the skin. In some embodiments, the method is directed to rejuvenating skin from a skin procedure comprising laser application to the skin.

In some embodiments, compounds of the disclosure are useful for improving or accelerating healing of skin grafting sites, such as on burns, scar revision, plastic surgery, or other procedures involving placement of a skin graft. As described elsewhere, the healing of the skin site from which a graft is take is also a benefit of the compounds described herein.

In some embodiments, compounds of the disclosure are useful in enhancing healing of a skin graft or a skin grafting site. In some embodiments, the skin grafting is provided to treat a burn. In some embodiments the burn is a partial-thickness burn. In some embodiments the burn is a full-thickness burn. In some embodiments, the skin grafting is provided to treat an injury, such as from a large open wound. In some embodiments, the skin grafting is provided to treat an ulcer such as but no limited to a bedsore. In some embodiments, the skin grafting is provided to treat a skin infection. In some embodiments, the skin grafting is provided to treat a skin cancer surgery site. In some embodiments, the skin grafting is provided to cover a larger surface area than available from the supply of donor skin.

In some embodiments, a method of enhancing hair follicle growth in skin comprises directly administering to the skin an amount of an inhibitor of fidgetin-like 2 effective to enhance hair follicle growth in skin. In some embodiments, the method increases hair growth in skin.

### Treatment of the Heart

In an embodiment, the wound is a cardiac tissue wound. In an embodiment, the wound is a cardiovascular wound, for example resulting from a myocardial infarction. In some embodiments, a compound of the disclosure promotes cardiac angiogenesis. In some embodiments, a compound of the disclosure improves cardiac function post myocardial infarction.

### Treatment of the Nervous System

In an embodiment, the wound is a neuronal wound.

In an embodiment, the wound is a wound of the central nervous system. In some embodiments, the wound in a spinal cord injury. In some embodiments, the prevention, reduction or inhibition of scarring may enhance neuronal reconnection and/or neuronal function. In some embodiments, a compound of the disclosure promotes nerve growth. In some embodiments, a compound of the disclosure reduces neuronal inflammation. In some embodiments, a compound of the disclosure promotes recovery from nerve transection. In some embodiments, a compound of the disclosure promotes nerve regeneration after injury. In some embodiments, the wound is a wound of the peripheral nervous system. In some embodiments the wound is a cavernous nerve injury. In some embodiments, the prevention, reduction or inhibition of scarring may enhance neuronal reconnection and/or neuronal function. In some embodiments, a compound of the disclosure promotes peripheral nerve growth. In some embodiments, a compound of the disclosure reduces neuronal inflammation. In some embodiments, a compound of the disclosure promotes recovery from nerve transection. In some embodiments, a compound of the disclosure promotes nerve regeneration after injury. In some embodiments, a compound of the disclosure has anti-inflammatory activity in neuronal and other tissues.

In some embodiments, a compound of the disclosure treats or prevents neuropraxia.

In some embodiments, a compound of the disclosure treats or prevents adverse sequelae of nerve sparing surgery.

In some embodiments, a compound of the disclosure promotes recovery of erectile response after unilateral or bilateral cavernous nerve transection. In some embodiments, a compound of the disclosure recovery of erectile response within two weeks of cavernous nerve injury. In some embodiments, cavernous nerve injury is a result of a surgical procedure such as prostatectomy. In some embodiments, prostatectomy is radical prostatectomy. In some embodiments, a wafer comprising a siRNA of the disclosure is implanted at the site of surgery. In some embodiments, siRNA concentrations of about 6.6, about 13.3 or about 26.6 micrograms per 100 mg wafer is implanted. In some embodiments, the wafer comprises about 2.5% collagen, about 7.5% chondroitin sulfate, about 82.5% polyvinylpyrrolidone, and about 7.5% polyethylene glycol 400.

### Treatment of the Eye

In an embodiment, the wound is a wound of the eye (including the inhibition of scarring resulting from eye surgery such as LASIK surgery, LASEK surgery, PRK surgery, glaucoma filtration surgery, cataract surgery, or surgery in which the lens capsule may be subject to scarring) such as those giving rise to corneal cicatrisation; wounds subject to capsular contraction (which is common surrounding breast implants).

### Treatment of the Vasculature

In an embodiment, the wound is a wound of the circulatory system, such as but not limited to a blood vessel, venous or arterial valves, heart valves, or enhancing the integration of a replacement heart valve, bypass graft, vasculature of a transplanted organ, by way of non-limiting examples.

### Treatment of the Musculoskeletal System

In an embodiment, the wound is a wound of tendons, ligaments or muscle.

### Treatment of the Oral Cavity

In an embodiment, the wound is a wound of the oral cavity, including the lips and palate. In some embodiments, the method inhibits scarring resulting from treatment of cleft lip or palate.

### Treatment of Organs and Cavities

In an embodiment, the wound is a wound of an internal organ such but not limited to the liver, heart, brain, digestive tissues and reproductive tissues.

In an embodiment, the wound is a wound a body cavity such as but not limited to the abdominal cavity, pelvic cavity and thoracic cavity. In some embodiments, inhibition of scarring may reduce the number of incidences of adhesion formation and/or the size of adhesions formed.

### Treatment of Surgical Wounds

In an embodiment, the wound is a surgical wound, such as but not limited to particular wounds associated with cosmetic procedures, such as scar revision. It is particularly preferred that the medicaments and methods of the disclosure be used to promote healing of wounds of the skin. Other non-limiting examples include surgical procedures to the eye and other parts of the body. As noted herein, the compound or composition of the disclosure may be applied to a site before the injury or wound occurs, such as a surgical incision.

### Other Aspects of the Disclosure

In an embodiment of the methods and compositions described herein the subject is a mammal. In an embodiment the subject is human.

As used herein, “promotion” of wound healing, or grammatical equivalent, means an acceleration in any one or more of visual appearance of wound recovery, reduction in wound size, reduction in distance between wound margins, scab formation, fibroplasia and re-epithelialization as compared to the corresponding parameter in an untreated wound.

As used herein, “wound” is a break or discontinuity in the structure of an organ or tissue (including skin), which includes epithelium, connective tissue, and muscle tissue, caused by an external agent. Examples of wounds include, but are not limited to, skin wounds, ulcerations, bedsores, grazes, tears, cuts, punctures, tympanic membrane perforations, burns, and those that are a consequence of plastic surgery procedures.

### Methods of Administration

The benefits that may be derived from the present disclosure may be applicable to wounds at any site throughout the body. In some embodiments, the wound for which healing is promoted is a skin wound. For illustrative purposes, the embodiments of the disclosure will generally be described with reference to skin wounds, although they remain applicable to other tissues and organs. Merely by way of example, in another preferred embodiment the wound may be a wound of the circulatory system, particularly of a blood vessel. Other wounds in which wound healing may be promoted in accordance with the present disclosure include as a result of surgery or as a result of a burn. Other wounds in which wound healing may be promoted in accordance with the present disclosure include skin ulcers caused by pressure, venous stasis, or diabetes mellitus. In some embodiments, the result of a wound is a scar, which may be treated as described herein to prevent or reduce scarring of a wound at any site in or on the body.

In an embodiment, the inhibitor of fidgetin-like 2 is administered locally to the wound. In an embodiment, the inhibitor of fidgetin-like 2 is administered via a vein or artery. In an embodiment, the inhibitor of fidgetin-like 2 is administered by injection, catheterization or cannulation. In an embodiment, the inhibitor of fidgetin-like 2 is administered from an implant that elutes the inhibitor, for example an eluting stent or an eluting skin patch.

The dosage of the inhibitor administered in treatment will vary depending upon factors such as the pharmacodynamic characteristics of a specific inhibitor and its mode and route of administration; the age, sex, metabolic rate, absorptive efficiency, health and weight of the recipient; the nature and extent of the symptoms; the kind of concurrent treatment being administered; the frequency of treatment with the inhibitor and the desired therapeutic effect.

A dosage unit of the inhibitor may comprise a single compound, or a mixture of the compound with one or more anti-infection compound(s) or wound healing-promoting compound(s).

In some embodiments, the inhibitor of fidgetin-like 2 is applied to the wound once. In some embodiments, the inhibitor of fidgetin-like 2 is applied to the wound more than once. In some embodiments, the inhibitor of fidgetin-like 2 is applied to the wound in the form of an controlled delivery device such as but no limited to a stent, wafer, implant, bandage, or any other slow release device. In some embodiments, the inhibitor of fidgetin-like 2 is applied to the wound each time the dressing is changed.

In some embodiments, the inhibitor of fidgetin-like 2 is applied to the wound until healing occurs. In some embodiments, the inhibitor of fidgetin-like 2 is applied to or maintained at the site for 1, 2, 3, 4, 5, 6, 7, 8, 9, 10, 11, 12, 13 or 14 days. In some embodiments, the inhibitor of fidgetin-like 2 is implanted or placed in a surgical site at the time of surgery. In some embodiments such placement is in the form of a controlled release composition such that the inhibitor of fidgetin-like 2 can act at the site for a period of time.

All combinations of the various elements described herein are within the scope of the disclosure unless otherwise indicated herein or otherwise clearly contradicted by context.

The following numbered embodiments, while non-limiting, are exemplary of certain aspects of the disclosure:
1. A nucleic acid molecule consisting of a sequence selected from the group consisting of:

(SEQ ID NO: 17)
5′-fUfUmA fCmAfC AGU AUU AAA GCG ATT;
(SEQ ID NO: 18)
(Phos) 5′-U CGC UUU AAU ACU G UG UAA TT;
(SEQ ID NO: 34)
5′-UUACACAGUAUUAAAGCGATT-3′;
(SEQ ID NO: 35)
(Phos) 5′-mUmCGCUUUAAUACUGUGUAATT-3′;
(SEQ ID NO: 36)
(Phos) 5′-mU(s)mC(s)GCUUUAAUACUGUGUAATT-3′;
(SEQ ID NO: 37)
(Phos) 5′-fUfCGCUUUAAUACUGUGUAATT-3′;
(SEQ ID NO: 38)
(Phos) 5′-fU(s)fC(s)GCUUUAAUACUGUGUAATT-3′;
(SEQ ID NO: 39)
(Phos) 5′-mU(s)mC(s)GCUUUAAUAmCfUmGfUmGfUmAmAT
T-3′;
(SEQ ID NO: 40)
(Phos) 5′-U(s)CGCUUUAAUACUGUGUAATT-3′;
(SEQ ID NO: 41)
(Phos) 5′-mUfCmGfCmUfUmUAAfUmAfCmUGmUmGfUmAmATT;
(SEQ ID NO: 42)
5′-mUmUmAmCmAmCmAmGmUmAmUmUmAmAmAmGmCmGmAmUmU-3′;
(SEQ ID NO: 43)
(Phos) 5′-mUmCmGmCmUmUmUmAmAmUmAmCmUmGmUmGmUmAmAm
UmU-3′;
(SEQ ID NO: 44)
5′ mUmUmAmCmAmCmAmGmUmAmUmUmAmAmAmGdCdGdATT-3′;
(SEQ ID NO: 45)
5′ mUmUmAmCmAmCmAmGmUmAmUmUmAmAmAmGdCmGmATT-3′;
(SEQ ID NO: 46)
5′ UUACACAGUAUUAAAGCGA-3′;
(SEQ ID NO: 47)
(Phos) 5′-U(s)CGCUUUAAUACUGUGUAATT-3′;
(SEQ ID NO: 48)
(Phos) 5′-UCGCUUUAAUACUGUGUAATT-3′;
(SEQ ID NO: 49)
(Phos) 5′-U(s)C(s)GCUUUAAUACUGUGUAATT-3′;
(SEQ ID NO: 50)
5′-mUmUACACAGUAUUAAAGCGA-3′;
(SEQ ID NO: 51)
(Phos) 5′-U(s)CGCUUUAAUACUGUGUmAmATT-3′;
(SEQ ID NO: 52)
(Phos) 5′-UCGCUUUAAUACUGUGUAATT-3′;
(SEQ ID NO: 53)
(Phos) 5′-U(s)C(s)GCUUUAAUACUGUGUAA T(s)T-3′;
(SEQ ID NO: 54)
5′ lUlUlAlClACAGUAUUAAAGCGATT-3′;
SEQ ID NO: 55)
(Phos) 5′-UCGCUUUAAUACUGlUlGlUlAlA TT-3′;
(SEQ ID NO: 56)
5′ fUfUlAfClACAGUAUUAAAGCGA-3′;
or
(SEQ ID NO: 57)
(Phos) 5′-mU(s)mCmGCUUUAAUACUGUGUAATT-3′,
wherein d(nucleotide) = deoxy-(nucleotide),
m(nucleotide) = 2′-O-methyl nucleotide, T =
thymidine, f(nucleotide) = 2′-fluorodeoxy
nucleotide, (Phos) = phosphodiester cap; capital
letter nucleotide = RNA nucleotide, ,
l(nucleotide) = a locked nucleotide, and (s) =
phosphorothioate.

2. A double stranded nucleic acid consisting of two nucleic acid molecules of embodiment 1.
3. A double stranded nucleic acid of embodiment 2 consisting of a sense strand selected from SEQ ID NOs: 17, 34, 42, 44, 45, 46, 50 and 54; and an antisense strand selected from SEQ ID NOs: 2, 18, 35, 36, 37, 38, 39, 40, 41, 43, 47, 48, 49, 51, 52, 53, 55 and 57.
4. A double-stranded nucleic acid of embodiment 2 consisting of SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; or SEQ ID NO:56 and SEQ ID NO:57.
5. A double stranded nucleic acid comprising a nucleic acid molecule of embodiment 1.
6. The double stranded nucleic acid of embodiment 5 wherein each strand has no more than 52 nucleotides.
7. The double stranded nucleic acid of embodiment 5 consisting of a sense strand comprising a nucleic acid molecule selected from among SEQ ID NOs: 17, 34, 42, 44, 45, 46, 50 and 54; and an antisense strand comprising a nucleic acid molecule selected from among SEQ ID NOs: 2, 18, 35, 36, 37, 38, 39, 40, 41, 43, 47, 48, 49, 51, 52, 53, 55 and 57.
8. The double-stranded nucleic acid of embodiment 5 comprising SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; or SEQ ID NO:56 and SEQ ID NO:57.
9. The double stranded nucleic acid of embodiment 5 wherein each strand has no more than 52 nucleotides.
10. The nucleic acid molecule of any one of embodiments 1-9, wherein at least one nucleotide is modified or further modified.
11. The nucleic acid of embodiment 10 wherein the modified or further modified nucleotide is selected from 2′-O-methyl-adenosine, 2′-O-methyl-uridine, 2′-O-methyl-cytosine, 2′-O-methyl-guanosine, 2′-O-methyl-thymidine, 2′-fluoro-adenosine, 2′-fluoro-cytidine, 2′-fluoro-guanosine, 2′-fluoro-uracil, 2′-fluoro-thymidine, deoxycytosine, deoxyguanosine, deoxyadenosine, deoxythymidine, deoxyuridine, a locked adenosine, a locked uridine, a locked guanosine, a locked cytidine, a phosphorothioate, and a phosphodiester cap.
12. A composition comprising the nucleic acid molecule of any one of embodiments 1-11 and a pharmaceutically acceptable carrier, vehicle, excipient or diluent.
13. The composition of embodiment 12, wherein said carrier comprises at least one of the following: saline, a sugar, a polypeptide, a polymer, a lipid, a cream, a gel, a micelle material, a wafer and a nanoparticle.
14. The composition of embodiment 12, wherein said carrier comprises at least one of the following: a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a nucleic acid delivery vehicle, a ligand functionalized-hydrophilic polymer grafted polymer, and a ligand functionalized liposome.
15. The composition of embodiment 14, wherein the carrier comprises a cationic polymer-nucleic acid complex.
16. The composition of embodiment 14, wherein the hydrophilic polymer is polyethylene glycol (PEG).
17. The composition of embodiment 13, wherein the nanoparticle is a liposomal nanoparticle.
18. The composition of embodiment 17, wherein the liposome is further functionalized with at least one 2′ sugar modification.
19. A method of treating a wound or inhibition, reducing or preventing a scar in a subject comprising administering to the subject a therapeutically effective amount of the composition of embodiment 12.
20. The method of embodiment 19 wherein the wound or scar is of the skin, eye, central nervous system, peripheral nervous system, cardiac tissue, blood vessel, tendon, ligament, muscle, oral cavity, lips, palate, internal organs, surgical wounds, abdominal cavity, pelvic cavity or thoracic cavity.
21. The method of embodiment 20 wherein the wound or scar of the eye is of the cornea or lens capsule.
22. The method of embodiment 20, wherein the wound or scar results from eye surgery, LASIK surgery, LASEK surgery, PRK surgery, glaucoma filtration surgery, cataract surgery, and corneal cicatrisation.
23. The method of embodiment 19, wherein inhibition of scarring reduces the number of incidences of adhesion formation and/or the size of adhesions formed.
24. The method of embodiment 19, wherein the where the prevention, reduction or inhibition of scarring enhances neuronal reconnection and/or neuronal function.
25. The method of embodiment 20, wherein the cardiac tissue wound is from a myocardial infraction.
26. The method of embodiment 20, wherein the wound is a neuronal wound.
27. The method of embodiment 20, wherein the wound results in a capsular contraction.
28. The method of embodiment 20, wherein the wound is a surgical wound.
29. The method of embodiment 20, wherein the wound is from a cosmetic procedure or a scar revision.
30. A method of accelerating or improving the healing of a skin graft or skin grafting site in a subject comprising administering to the subject an amount of composition of embodiment 12 effective to accelerate healing of the skin graft or skin grafting site.

This disclosure will be better understood from the Experimental Details, which follow. However, one skilled in the art will readily appreciate that the specific methods and results discussed are merely illustrative of the disclosure as described more fully in the claims that follow thereafter.

### EXAMPLES

### Example 1. siRNA Synthesis

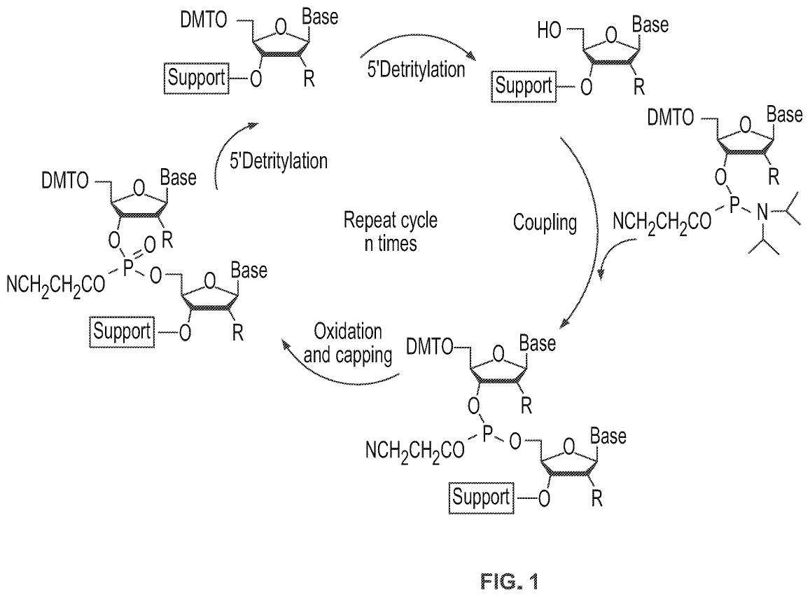

Oligonucleotide sequences SEQ ID NOs: 17-18 and 34-57 were prepared by synthesizing the two single strands of oligonucleotide (sense and antisense) by conventional solid-phase oligonucleotide synthesis using phosphoramidite chemistry. Assembly of an oligonucleotide chain by the phosphoramidite method on a solid support such as controlled pore glass (CPG) or polystyrene is shown in FIG. 1.

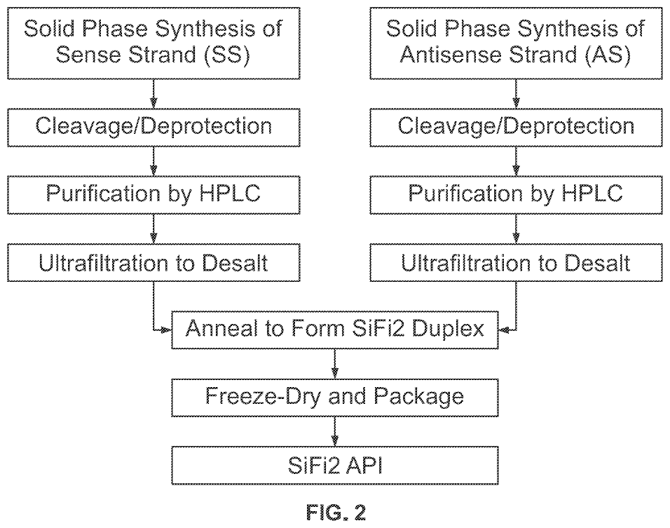

Each cycle consists of 5′ deprotection, coupling, oxidation, and capping. Each coupling step is carried out by reaction of the appropriate activated amidite with the free 5′ hydroxyl group of a support-immobilized protected nucleoside or oligonucleotide. The oligonucleotide is then deprotected and cleaved from the support. The 2′ TBDMS protecting group is then cleaved to yield the crude sense or antisense strand. The sense and antisense strands are then individually purified. The purified single strands are analyzed to confirm the correct molecular weight and impurity profile prior to annealing into the siRNA duplex (referred to as SiFi2 in FIG. 2). The annealed duplex is freeze-dried to yield the active pharmaceutical ingredient (API). The API is stored at −20° C.

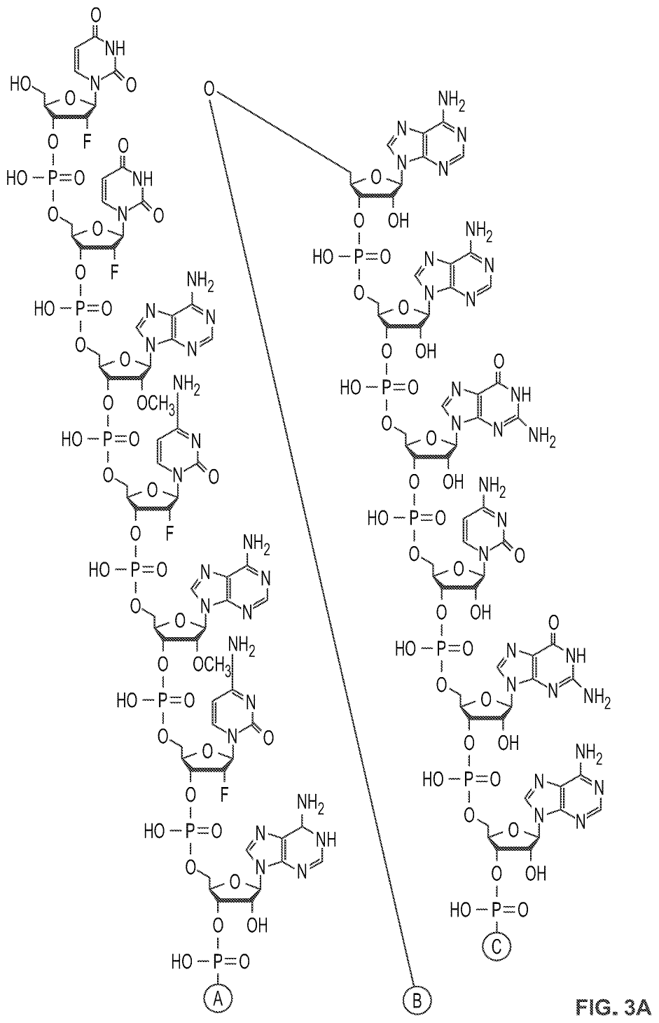

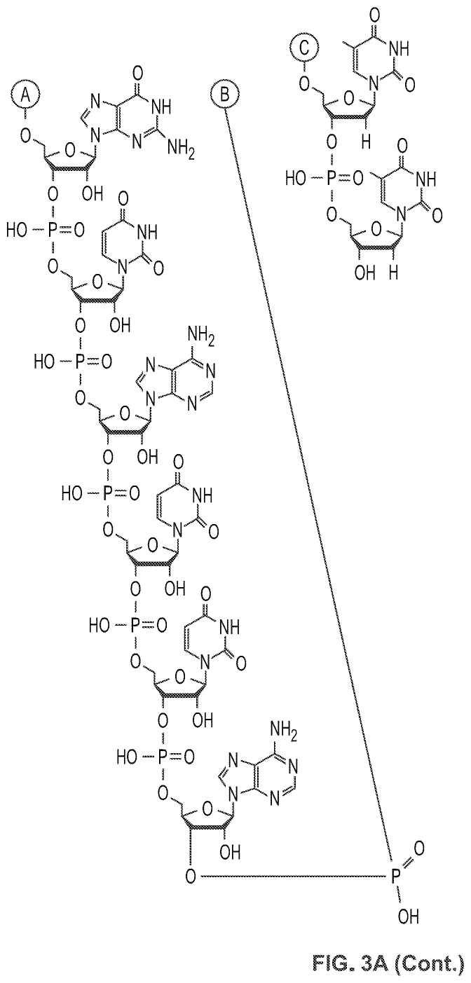

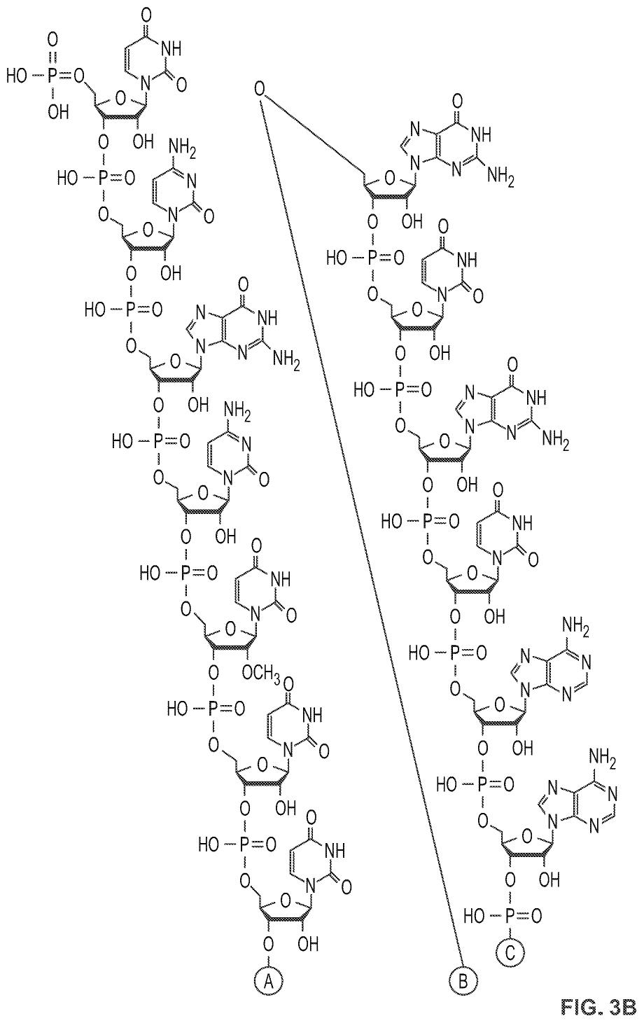

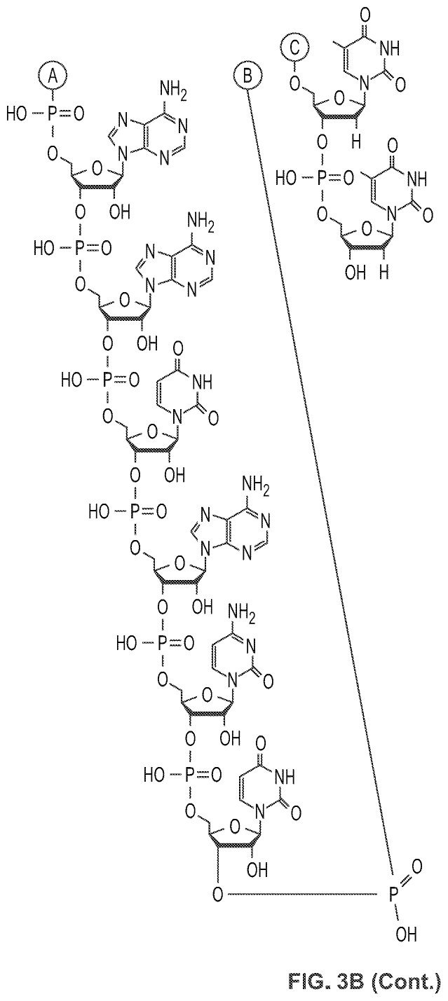

FIG. 3 depicts the chemical structure of one pair of oligonucleotides, sense nucleic acid molecule SEQ ID NO:17 (FIG. 3A) and antisense nucleic acid molecule SEQ ID NO:18 (FIG. 3B). The calculated mass, found mass (via mass spectrometry), and chemical formula of SEQ ID NO:17/18 is found in Table 1. The designation 17/18 refers to a duplex of SEQ ID NO:17 and SEQ ID NO:18.

TABLE 1

| | Exact | Average | |
|---|---|---|---|
| | Mass | Mass | |
| Compound | (Daltons) | (Daltons) | Chemical Formula |
| Sense Strand | 6696.98 | 6700.14 | C₂₀₄H₂₅₂F₄N₇₈O₁₃₈P₂₀ |
| (SEQ ID NO: 17) | | | |
| Antisense Strand | 6683.85 | 6686.98 | C₂₀₀H₂₅₀N₆₉O₁₅₁P₂₁ |
| (SEQ ID NO: 18) | | | |
| SiFi2 Duplex | 13380.83 | 13387.12 | C₄₀₄H₅₀₂F₄N₁₄₇O₂₈₉P₄₁ |
| (SEQ ID | | | |
| NO: 17/18) | | | |

The other nucleic acid molecules described herein were prepared in the same manner.

### Example 2. siRNA Transfection of U2OS Cells

The following methods are used to transfect U2OS cells in preparation for testing the efficacy of nucleic acids and siRNAs described herein.

siRNA transfection protocol (6 well plate). Seed 100,000 U2OS cells per well (6 well dish) and culture for 2 days (−80% confluency). The cells should be serum free media 12 hours before transfection. Lipofectamine RNAiMAX: Dilute 3.5 μL of siRNA (20 μM stock)/transfection (70 μmol) into 250 μL OptiMEM. Then dilute 3.5 μL of Lipofectamine 3000 into 250 μL of OptiMEM. Mix siRNA/OptiMEM into Lipofectamine/OptiMEM solution. Incubate for 5 minutes at room temperature. Add mixture dropwise to wells. Add 500 μL of serum free media.

siRNA transfection protocol (24 well plate). Seed 20,000 U2OS cells per well (6 well dish) and culture for 2 days (−80% confluency). Follow Lipofectamine 3000 protocol. Dilute 0.7 μL of siRNA (20 μM stock)/transfection (70 μmol) into 125 μL OptiMEM. Dilute 0.7 μL of Lipofectamine 3000 into 125 μL of OptiMEM. Mix siRNA/OptiMEM into Lipofectamine/OptiMEM solution. Incubate for 15 minutes at room temperature. Add mixture dropwise to wells. Add 250 μL of serum free media.

### Example 3. siRNA Nucleofection of HUVECs

The following nucleofection protocol was followed for HUVECs.

Solution 1: 125 mM Na₂HPO₄ (1.4998 g in 100 ml H₂O), 12.5 mM KCl (0.09318 g in 100 mL H₂O). Solution 2: 55 mM MgCl₂ (0.9524 g in 100 mL H₂O). Working solution: 80% solution 1, 20% solution 2. Use 100 μL per nucleofection.

Protocol. Have warm M200 media in plates ready in the incubator. Select HUVEC Nucleofection protocol (CM-104 for U2OS) on Nucleofector before starting. Best if cells have been split 48 hours prior to nucleofection. Work as quickly as possible, minimize the amount of time cells spend in nucleofection solution.

Procedure: Trypsinize 1 T75 of HUVECs and count. Usually ˜1.3 million cells/flask. Pellet cells at 1200 rpm for 5 mins. Resuspend pellet in nucleofection solution and evenly split between the number of Nucleofection cuvettes. 100 μL/nucleofection. Nucleofect between 1×10⁴-1×10⁶ cells per cuvette. Add nucleic acid to be nucleofected directly to cuvette (30 pmol siRNA=1.5 μL of 20 μM stock siRNA/1-2 ug plasmid DNA). Flick sides to remove any bubbles. Place cuvettes into Nuclefector and run. Add 500 μL warm media directly to cuvettes. Plate cells into well plates filled with warm media waiting. Swirl plates to even distribute cells. Analyse knockdown/gene expression 24 to 48 hours later.

### Example 4. Efficacy Testing—Migration Assay

U2OS cells were seeded into wells (100,000 cells per well) on a 6-well dish and cultured for two days until reaching ˜80% confluency. As described above, cells were transfected with 3.5 μL of 20 μM siRNA (70 pmol) using 3.75 μL Lipofectamine 3000, following the manufacturer's protocol. After 24 hours cells were washed and grown until harvest at either 24, 48, or 72 hours.

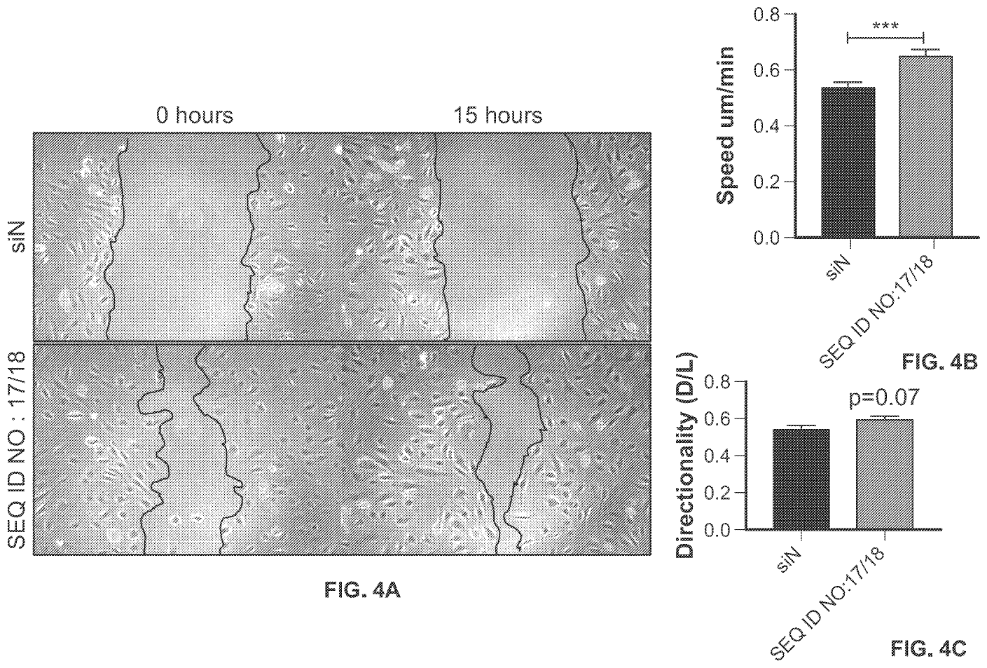

Migration of U2OS cells and HUVEC comprising siRNA as described above were monitored using time-lapse phase-contrast images showing representative results from scratch assays performed with U2OS cells after treatment with control non-targeting siRNA (siN) or FL2 siRNA. FIG. 4A shows an example of images taken during the assay; scale bar is 100 μm. (4B) Graphs comparing the average migration rate in control and FL2-depleted cells. Data were accumulated from three independent scratch assay experiments. (4C) Graphs comparing the directional persistence of migrating control vs. FL2-depleted cells. Directionality was determined as the distance (D) between the start and endpoints divided by the total path length (L) of each trajectory. Error bars show Standard Error of the Mean (SEM). ***P<0.05.

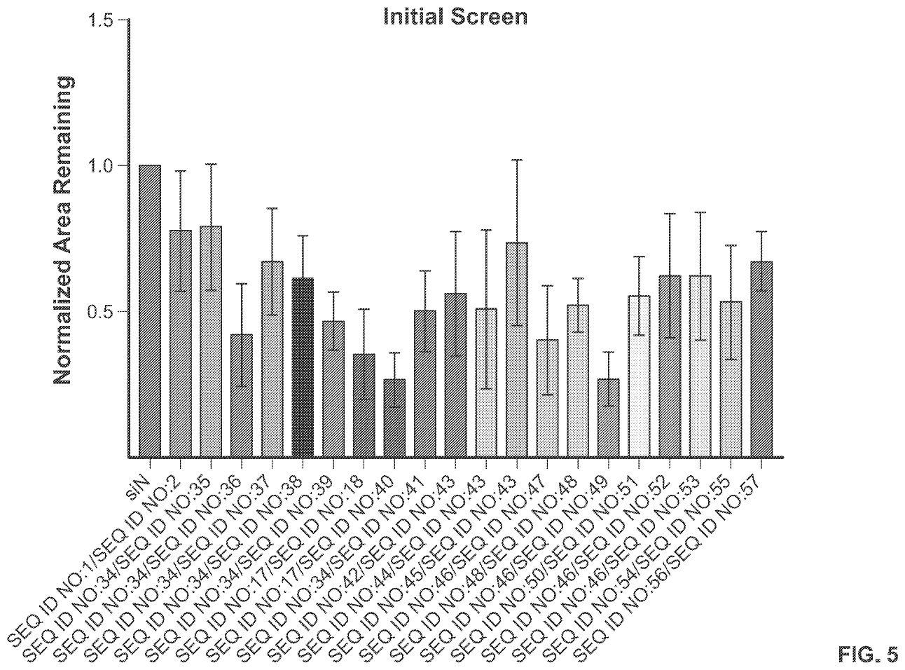

Using the same assay, the following double-stranded siRNA from among SEQ ID NOs:17-57 were tested to demonstrate that these siRNAs impacted cellular phenotypes. The siRNA tested were SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; SEQ ID NO:56 and SEQ ID NO:57; U2OS were transfected as described above with each of the siRNAs and grown to confluency. Wells were then scratched and imaged over the course of healing. Data are shown in FIG. 5 of an initial screen in U2OS cells.

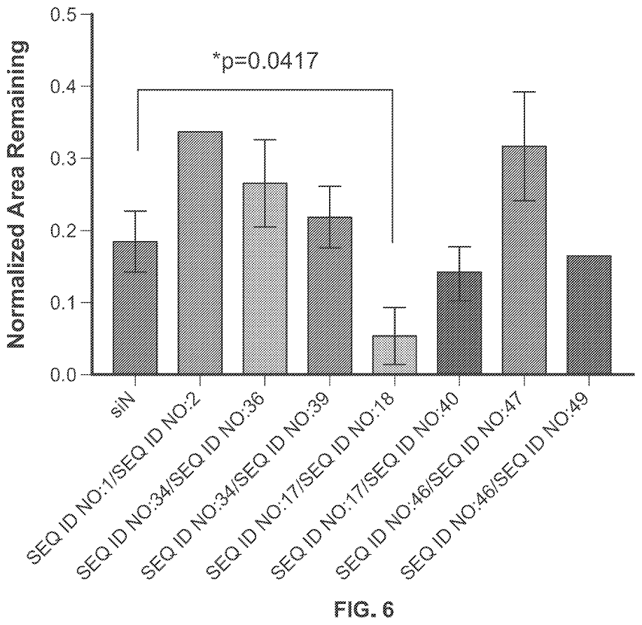

Certain candidates identified in the initial screen were tested in larger samples sizes n=4, as shown in FIG. 6.

HUVEC cells were nucleofected as described above with either control siRNA, or FL2 targeting siRNA, plated and allowed to reach confluence before performing a time-lapse scratch assay. Individual cells were tracked in using FIJI and cell migration speed was analyzed using the Diper Excel macros. Student's t test was performed to determine significant differences. n≥49 cells. Cells were harvested for Western Blot and probed to confirm siRNA mediated FL2 knockdown. GAPDH was used as a loading control.

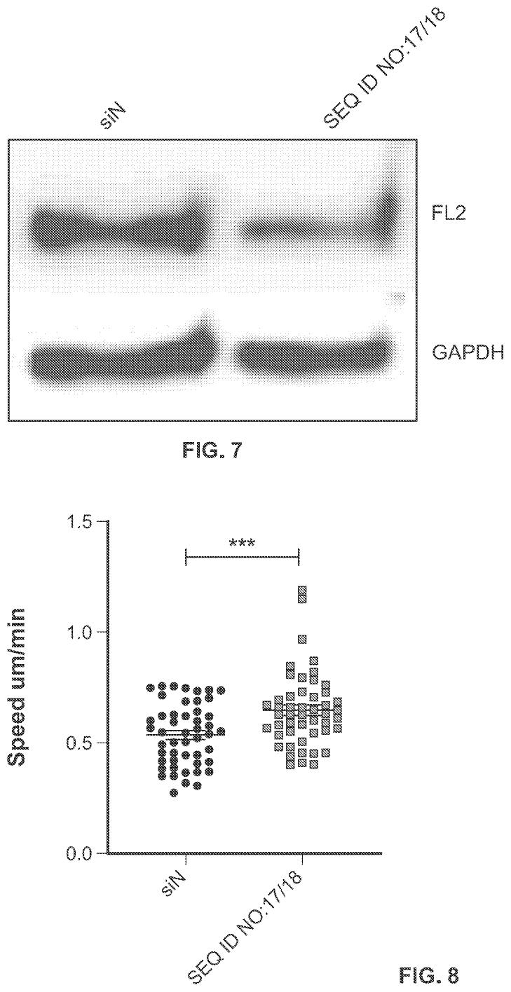

Cell tracking indicated that these modifications improved cell migration speed while effectively producing a knockdown. Knock-down results for siRNA comprising SEQ ID NOs: 17/18 is shown in FIG. 7, and migration data in FIG. 8.

In vitro, depletion of FL2 by SEQ ID NO:17/18 siRNA from human tissue culture cells results in an increase in the rate of cell movement, due in part to an increase in directional motility. Other siRNA sequences comprising a nucleic acid molecule from among SEQ ID NO:17-57 also show an increase in migration speed.

### Example 5. siRNA Compositions

Nanoparticles (np) comprising a siRNA of the disclosure were formulated using five hundred μl of tetramethyl orthosilicate (TMOS) was hydrolyzed in the presence of 100 μl of 1 mM HCl by sonication on ice for about 15 min, until a single phase formed. The hydrolyzed TMOS (100 μl) was added to 900 μl of 20 μM of siRNA (mouse FL2 (Sigma-Aldrich, SASI_Mm02_00354635) or the negative control) solution containing 10 mM phosphate, pH 7.4. A gel was formed within 10 minutes. The gel was frozen at −80° C. for 15 minutes and lyophilized.

A wafer comprising siRNA of the disclosure is made from 2.5% collagen, 7.5% chondroitin sulfate, 82.5% polyvinylpyrrolidone, and 7.5% polyethylene glycol 400. Such wafers are made to contain 6.6, 13.3 or 26.6 micrograms siRNA per 100 mg wafer. A wafer is implanted at a surgical site, such as during nerve-sparing surgery or procedures with high risk of neuronal dysfunction such as a radical prostatectomy.

### Example 6. Nanoparticle Microemulsion Formulation

Constituents needed for the methodology: Zonyl FSO-100 (FSO), Poloxamer 188, Perfluorodecalin (PFD), DNAse/RNAse free water, siRNA as described herein, or Control.

Pre-procedure precautions before starting the formulation. The protocol is performed in a sterile environment at room temperature using DNAse/RNAse free water. The containers necessary to process the formulation is pre-cleaned with RNAse zap, autoclaved and rinsed with DNAse/RNAse free water in a sterile laminar hood.

Preparation of organic phase. A 20% solution of PFD is made in FSO in a tissue culture laminar hood. For a batch formulation of 100 mL, weigh 20 grams of FSO and add PFD to make the volume to 80 mL. The mixture was vortexed and sonicated in an ultrasonic water batch alternatively every 10 min for 1 h followed by 4 h stirring. Periodically the mixture should be checked for consistency since the FSO is sparingly soluble in PFD. In order to avoid big chunks of FSO and entrapment of air, the mixture should be sonicated in an ultrasonic water batch making sure not to expose the mixture to moisture or water. The solubility could take somewhere between overnight stirring and 24 hrs at room temperature. If large chunks of undissolved FSO is present it should be separated from the mixture by slow centrifugation @300 g for 5 minutes at room temperature. The dissolved phase should be carefully decanted into sterile falcon tubes. Keep the supernatant aside, and add 10 mL of PFD to the larger chunks of FSO, vortex the mixture with the cap tightly closed followed by sonication in an ultrasonic water bath. The procedure should be repeated until the larger chunks of PFD are completely dissolved in PFD. Pool both the PFD mixtures to make it to 90 mL. This should result in a homogeneous suspension.

An alternative scale-up process can be done by preparing the organic phase in smaller quantities and then pool all the fractions in the end to obtain a homogeneous suspension.

Characterization of particle size in the organic phase. The mixture of PFD should be of a specific particle size preferably below 5 microns. After overnight stirring of the FSO in PFD, an aliquot (10 μL) of the mixture diluted and subjected to dynamic light scattering (DLS) to monitor the particle size optimized to have maximum stability. At higher particle sizes the stability of the formulation is less viable and to have the maximum efficiency, it is better to have the particle size around or less than 5 micrometers. If the particle size is larger than 5 microns by DLS measurements, an additional step of sonication using a probe is performed with slow pulse with 20 sec interval. Care should be taken not to exceed the sonication procedure for more than 10 minutes. If there are still larger particles more than 5 microns by DLS measurements, the mixture should be stirred overnight under sterile conditions to have a mixture of uniform particle size.

DLS Result: 3.433±0.215 microns. Instrument: Brookhaven Instruments Corporation, particle size analyzer

Preparation of aqueous phase. In a separate 50 mL falcon tube, prepare 4% of Poloxamer solution in DNAse/RNAse free water. Weigh 400 mg of Poloxamer in 9 mL of DNAse/RNAse free water and mix in a nutator for 2 hours checking for consistency every 30 minutes. Poloxamer should dissolve completely in DNAse/RNAse free water. The Poloxamer solution should be chilled in an ice water bath till use or refrigerated. The Poloxamer solution should be made in the clean laminar hood and could be mixed using a nutator after tight capping outside the hood. (Note: The Poloxamer solution is made in 9 mL of water and later 1 mL of siRNA mixed in DNAse/RNAse free water is added to make the aqueous phase of Poloxamer).

Addition of the siRNA/control in the aqueous phase. The siRNA powder or liquid is mixed in pre-chilled DNAse/RNAse free water and made up to 1 mL. The siRNA is quickly thawed and diluted with DNAse/RNAse free water in an ice bucket just before making the formulation. Do not thaw the siRNA until everything is ready for the formulation. Gently add the 1 mL of siRNA solution to the 9 mL of pre-chilled Poloxamer solution in the laminar hood, and mix well to obtain homogeneous solution. This procedure should be performed in a certified biosafety hood.

Preparation of the emulsion formulation. Stir the PFD mixture at a constant speed of 1200 rpm using a magnetic stir bar inside the laminar hood on an ice bath. The consistency and stability of the micro emulsion formation is checked by an optical microscope periodically. Gently add the aqueous phase of Poloxamer and siRNA mixture slowly using a micropipette. The slow addition of the aqueous phase is critical and faster addition may result in separation of the organic and the aqueous phase. The total 10 mL of the aqueous phase is added over a period of 20 minutes or more at the rate of 0.5 mL/minute or less to obtain a stable emulsion. The stability of the emulsion should be tested by monitoring the phase separation while the solution stands for 1 h at 4° C.

Characterization of emulsion formulation Microscopic image. DLS Result: 4.358±0.657 microns. Instrument: Brookhaven Instruments Corporation, particle size analyzer

Zeta potential Result: −16.97±2.09 mV. Instrument: Brookhaven Instruments Corporation, PALS zeta potential analyzer

Emulsion stability (1 h)=100%;

Using (Vb−Va)/Vb×100%; wherein Vb is the volume of the aqueous phase before emulsification; Va is the volume of the aqueous phase after emulsification

pH: 6.0

Charge: Neutral

Storage and use. Store the emulsion or the spray formulation at 4□C until use (do not freeze). The emulsion should be shaken well before use.

### Example 7. siRNA Collagen—Surfactant Polymer Dressing

A dressing for treating wounds, burns and other injuries using a collagen microparticle and surface polymer dressing (SPD) is made as follows: 10 g of sodium bis(2-ethylhexyl) sulfosuccinate (AOT) (Sigma-Aldrich) is dissolved in 34 ml of n-hexane and 2 ml of 5% collagen-I dissolved in acetic acid is added. The resulting microemulsion is stirred for 45 min until it becomes clear. This solution is then evaporated to remove the hexane. The residue is washed and is then suspended in nuclease free water and lyophilized. The 100 mg of lyophilized powder is then treated with 1000 μl of 25 μM siRNA solution and re-lyophilized. This material is then suspended in 1.25 mL of SPD, at 4 degrees for 2 hours, and is then lyophilized. The lyophilized powder is then added with 1.25 mL of nuclease free water and 1.25 mL of SPD.

### Example 8. siRNA Improves Outcome of Radical Prostatectomy

A radical prostatectomy is performed on a prostate cancer patient. Such surgeries may have an up to 50% risk of erectile dysfunction. To reduce the possibility of erectile function and other neurologic complications post-surgery, a 100 mg wafer prepared as described herein comprising 10 micrograms of siRNA of SEQ ID NOs:17/18 is implanted at the surgical site proximal to the cavernous nerves. The patient recovers erectile function post-surgery.

### Example 9. siRNA Improves Excisional Wound Healing

A double blind, placebo controlled, randomized excisional wound clinical trial in normal healthy volunteers is conducted to evaluate the rate of wound healing in split thickness skin graft (STSG) donor sites. In normal volunteers, a 0.08 inch thickness STSG of dimension one inch by one inch will be taken using a calibrated microdermatome from the upper outer aspect of each buttock. Subjects will receive initial hemostasis management using standard techniques (pressure, thrombin spray, epinephrine). A wound photograph will be taken to fill 80% of the camera frame with a calibration ruler within the field of the photo. Using a side by side randomization designation, a Telfa® pad saturated with fixed dose of SEQ ID NOs: 17/18 will be applied to one side STSG donor site. In addition, a Telfa® pad saturated with the vehicle will be administered to the opposite side. A sterile, non-adhesive film will be placed over the Tegaderm™, followed by a gauze bolster that will be taped in place. During repeat dosing, the dressing will be taken down to include the film but not the Telfa pad. Repeat doses will be used to saturate the Telfa pad, as did the first dose. The dressing will then be restored, as above. One day after the final dose is administered, the bolster will be removed, and both Telfa® pads will be gently soaked away from the donor sites using saline irrigation. A second, similar photograph will be taken. The wound will then be dried and covered with a transparent, breathable filmic dressing, allowing visualization of wound healing thereafter. Photographs will be taken daily for two weeks or until complete epithelialization has occurred. The filmic dressing will be removed when the clinician deems that 100% wound epithelialization has occurred or if otherwise clinically indicated. Photographic planimetry will be performed by a blinded observer and rates of wound healing at all time points and time to complete epithelialization will be measured and reported.

Subjects will return for photographs at one, three, and six months to ascertain durability of healing and quality of scar using Category 1 of the Hamilton Scar Assessment Scale.

Frequency of dosing (qd, bid, and tid) will be explored among three cohorts.

Primary Objective: Demonstrate that STSG donor site treatment with SiFi2 supports more rapid wound healing than STSG donor site areas treated with vehicle alone.

Secondary Objectives: Demonstrate that wound healing after treatment of STSG donor sites with SiFi2 endures and is not associated with hypertrophic scarring as determined by the Hamilton Scar Assessment Scale, as compared to STSG donor site areas treated with vehicle.

Primary Endpoint: Rate and completion of STSG donor site wound healing, as determined by interpretation of standardized photography at Days 5-19.

Secondary Endpoints: (1) Maintenance of healed wound at one, three, and six months; (2) Degree of hypertrophic scarring in each treatment arm as assessed by Category 1 of the Hamilton Scar Assessment Scale6 score on photographs of the STSG donor sites at one, three, and six months as interpreted by three independent expert wound care surgeon reviewers; (3) Degree of pain and pruritus on the treated and untreated sides.

Inclusion Criteria: (1) Male and female healthy subjects of all races; (2) Age Range: 21-65 inclusive; (3) Basal Metabolic Index between 18 and 30; (4) Willing and able to provide Informed Consent and to participate in scar evaluation postoperatively; (5) Willingness to adhere to the follow up evaluation schedule. Exclusion Criteria: (1) Inability to provide Informed Consent; (2) Unwillingness to participate in scar evaluation postoperatively; (3) Cutaneous disease (scleroderma or other collagen vascular disease, prior keloid, severe skin thinning with prior skin tears); (4) The use of systemic steroids or dermatological steroids in the last six months; (5) Pregnancy or trying to become pregnant; (6) On anticoagulants; (7) Immune deficiency state; (8) Diabetes mellitus; (9) Malnourished; (10) Platelet or NSAID use in the prior two weeks; (11) Known hypersensitivity to suture or bandage materials; (12) Known hypersensitivity to epinephrine or thrombin; (13) Infection within the previous two weeks; (14) Any condition that in the opinion of the investigator will not allow the subject to successfully complete the clinical trial.

Safety: All adverse events, clinically significant laboratory abnormalities from baseline, abnormal bleeding, infection, and hypertrophic scar formation will be monitored.

Number of Subjects: Approximately 15 completed subjects across three cohorts (five subjects per cohort). One cohort would receive the treatment once per day (qd), the second cohort twice per day (bid), and the third cohort three times per day (tid).

Study Participation: Six months

Each subject would receive four days of drug applied on Telfa® absorptive pad beneath a tie over bolster at STSG donor sites either qd (cohort 1), bid (cohort 2), or tid (cohort 3) on Days 1, 2, 3, and 4. Total surgical time in any instance is estimated to be less than 2 hours.

Estimated Time to Complete Enrollment: Three months.

The results will show that STSG donor site treatment with SEQ ID NOs:17/18 supports more rapid wound healing than STSG donor site areas treated with vehicle alone, and that wound healing after treatment of STSG donor sites with SiFi2 endures and is not associated with hypertrophic scarring as determined by the Hamilton Scar Assessment Scale, as compared to STSG donor site areas treated with vehicle.

While certain features of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

1. A nucleic acid molecule consisting of a sequence selected from the group consisting of:
(SEQ ID NO: 17)
5′-fUfUmA fCmAfC AGU AUU AAA GCG ATT;
(SEQ ID NO: 18)
(Phos) 5′-U CGC UUU AAU ACU G UG UAA TT;
(SEQ ID NO: 34)
5′-UUACACAGUAUUAAAGCGATT-3′;
(SEQ ID NO: 35)
(Phos) 5′-mUmCGCUUUAAUACUGUGUAATT-3′;
(SEQ ID NO: 36)
(Phos) 5′-mU(s)mC(s)GCUUUAAUACUGUGUAATT-3′;
(SEQ ID NO: 37)
(Phos) 5′-fUfCGCUUUAAUACUGUGUAATT-3′;
(SEQ ID NO: 38)
(Phos) 5′-fU(s)fC(s)GCUUUAAUACUGUGUAATT-3′;
(SEQ ID NO: 39)
(Phos) 5′-mU(s)mC(s)GCUUUAAUAmCfUmGfUmGfUmAmAT
T-3′;
(SEQ ID NO: 40)
(Phos) 5′-U(s)CGCUUUAAUACUGUGUAATT-3′;
(SEQ ID NO: 41)
(Phos) 5′-mUfCmGfCmUfUmUAAfUmAfCmUGmUmGfUmAmATT;
(SEQ ID NO: 42)
5′-mUmUmAmCmAmCmAmGmUmAmUmUmAmAmAmGmCmGmAmUmU-3′;
(SEQ ID NO: 43)
(Phos) 5′-mUmCmGmCmUmUmUmAmAmUmAmCmUmGmUmGmUmAmAm
UmU-3′;
(SEQ ID NO: 44)
5′ mUmUmAmCmAmCmAmGmUmAmUmUmAmAmAmGdCdGdATT-3′;
(SEQ ID NO: 45)
5′ mUmUmAmCmAmCmAmGmUmAmUmUmAmAmAmGdCmGmATT-3′;
(SEQ ID NO: 46)
5′ UUACACAGUAUUAAAGCGA-3′;
(SEQ ID NO: 47)
(Phos) 5′-U(s)CGCUUUAAUACUGUGUAATT-3′;
(SEQ ID NO: 48)
(Phos) 5′-UCGCUUUAAUACUGUGUAATT-3′;
(SEQ ID NO: 49)
(Phos) 5′-U(s)C(s)GCUUUAAUACUGUGUAATT-3′;
(SEQ ID NO: 50)
5′-mUmUACACAGUAUUAAAGCGA-3′;
(SEQ ID NO: 51)
(Phos) 5′-U(s)CGCUUUAAUACUGUGUmAmATT-3′;
(SEQ ID NO: 52)
(Phos) 5′-UCGCUUUAAUACUGUGUAATT-3′;
(SEQ ID NO: 53)
(Phos) 5′-U(s)C(s)GCUUUAAUACUGUGUAA T(s)T-3′;
(SEQ ID NO: 54)
5′ lUlUlAlClACAGUAUUAAAGCGATT-3′;
SEQ ID NO: 55)
(Phos) 5′-UCGCUUUAAUACUGlUlGlUlAlA TT-3′;
(SEQ ID NO: 56)
5′ fUfUlAfClACAGUAUUAAAGCGA-3′;
or
(SEQ ID NO: 57)
(Phos) 5′-mU(s)mCmGCUUUAAUACUGUGUAATT-3′,
wherein d(nucleotide) = deoxy-(nucleotide),
m(nucleotide) = 2′-O-methyl nucleotide, T =
thymidine, f(nucleotide) = 2′-fluorodeoxy
nucleotide, (Phos) = phosphodiester cap; capital
letter nucleotide = RNA nucleotide, ,
l(nucleotide) = a locked nucleotide, and (s) =
phosphorothioate.

2. A double stranded nucleic acid consisting of two nucleic acid molecules of claim 1.

3. A double stranded nucleic acid of claim 2 consisting of a sense strand selected from SEQ ID NOs: 17, 34, 42, 44, 45, 46, 50 and 54; and an antisense strand selected from SEQ ID NOs: 2, 18, 35, 36, 37, 38, 39, 40, 41, 43, 47, 48, 49, 51, 52, 53, 55 and 57.

4. A double-stranded nucleic acid of claim 2 consisting of SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; or SEQ ID NO:56 and SEQ ID NO:57.

5. A double stranded nucleic acid comprising a nucleic acid molecule of claim 1.

6. The double stranded nucleic acid of claim 5 wherein each strand has no more than 52 nucleotides.

7. The double stranded nucleic acid of claim 5 consisting of a sense strand comprising a nucleic acid molecule selected from among SEQ ID NOs: 17, 34, 42, 44, 45, 46, 50 and 54; and an antisense strand comprising a nucleic acid molecule selected from among SEQ ID NOs: 2, 18, 35, 36, 37, 38, 39, 40, 41, 43, 47, 48, 49, 51, 52, 53, 55 and 57.

8. The double-stranded nucleic acid of claim 5 comprising SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:52; SEQ ID NO:46 and SEQ ID NO:53; SEQ ID NO:54 and SEQ ID NO:55; or SEQ ID NO:56 and SEQ ID NO:57.

9. The double stranded nucleic acid of claim 5 wherein each strand has no more than 52 nucleotides.

10. The nucleic acid molecule of claim 1, wherein at least one nucleotide is modified or further modified.

11. The nucleic acid of claim 10 wherein the modified or further modified nucleotide is selected from 2′-O-methyl-adenosine, 2′-O-methyl-uridine, 2′-O-methyl-cytosine, 2′-O-methyl-guanosine, 2′-O-methyl-thymidine, 2′-fluoro-adenosine, 2′-fluoro-cytidine, 2′-fluoro-guanosine, 2′-fluoro-uracil, 2′-fluoro-thymidine, deoxycytosine, deoxyguanosine, deoxyadenosine, deoxythymidine, deoxyuridine, a locked adenosine, a locked uridine, a locked guanosine, a locked cytidine, a phosphorothioate, and a phosphodiester cap.

12. A composition comprising the nucleic acid molecule of claim 1 and a pharmaceutically acceptable carrier, vehicle, excipient or diluent.

13. The composition of claim 12, wherein said carrier comprises at least one of the following: saline, a sugar, a polypeptide, a polymer, a lipid, a cream, a gel, a micelle material, a wafer and a nanoparticle.

14. The composition of claim 12, wherein said carrier comprises at least one of the following: a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a nucleic acid delivery vehicle, a ligand functionalized-hydrophilic polymer grafted polymer, and a ligand functionalized liposome.

15. The composition of claim 14, wherein the carrier comprises a cationic polymer-nucleic acid complex.

16. The composition of claim 14, wherein the hydrophilic polymer is polyethylene glycol (PEG).

17. The composition of claim 13, wherein the nanoparticle is a liposomal nanoparticle.

18. (canceled)

19. A method of treating a wound or inhibition, reducing or preventing a scar in a subject comprising administering to the subject a therapeutically effective amount of the composition of claim 12.

20. The method of claim 19 wherein the wound or scar is of the skin, eye, central nervous system, peripheral nervous system, cardiac tissue, blood vessel, tendon, ligament, muscle, oral cavity, lips, palate, an internal organs, a surgical wounds, abdominal cavity, pelvic cavity or thoracic cavity.

21.-29. (canceled)

30. A method of accelerating or improving the healing of a skin graft or skin grafting site in a subject comprising administering to the subject an amount of composition of claim 12 effective to accelerate healing of the skin graft or skin grafting site.
